(19) United States
(12) Patent Application Publication
(10) Pub. No.: US 2010/0272779 A1
(43) Pub. Date: Oct. 28, 2010

(54) PROGESTERONE-CONTAINING COMPOSITIONS AND DEVICES

(76) Inventor: Gregg A. Jackson, San Francisco, CA (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(21) Appl. No.: 12/791,818

(22) Filed: Jun. 1, 2010

Related U.S. Application Data
(63) Continuation-in-part of application No. PCT/US2008/085120, filed on Dec. 1, 2008.
(60) Provisional application No. 60/991,033, filed on Nov. 29, 2007.

(51) Int. Cl. A61F 2/82; A61K 31/57; A61K 9/127; A61P 35/00; A61P 7/02; A61P 9/10

(52) U.S. Cl. 424/423; 514/177; 424/450; 623/1.42

(57) ABSTRACT

Progesterone-containing compositions and devices that can maintain opening of a body passageway are described. One aspect of the invention provides a therapeutically effective (e.g., relaxative, anti-oxidative, anti-restenotic, anti-angiogenic, anti-neoplastic, anti-cancerous, anti-precancerous and/or anti-thrombotic) composition or formulation containing progesterone and optionally vitamin E and/or conjugated linoleic acid. Another aspect of the invention provides a drug eluting device, such as a drug eluting stent, with at least one coating layer comprising a progesterone composition that can minimize or eliminate inflammation, thrombosis, restenosis, neo-intimal hyperplasia, rupturing of vulnerable plaque, and/or other effects related to device implantation, treatment, or interaction. Other aspects of the invention provide for methods of using such compositions, formulations, and devices.

### CROSS-REFERENCE TO RELATED APPLICATIONS

This application is a Continuation-in-Part application claiming priority to PCT/US08/85120, filed Dec. 1, 2008, which in turn claims priority to U.S. Provisional Application Ser. No. 60/991,033, filed Nov. 29, 2007, each of which is incorporated herein by reference in its entirety.

### FIELD OF THE INVENTION

The present invention generally relates to anti-angiogenic, anti-thrombotic, anti-neoplastic, and/or anti-restenotic compositions, formulations, coated devices, and methods for their use.

### BACKGROUND

Implantable medical devices, such as stents, are widely employed in medical procedures. A stent is generally understood in the art to be an expandable prosthetic device for implantation in a body passageway (e.g., a lumen or artery) to keep a formerly blocked passageway open and/or to provide support to weakened structures (e.g. heart walls, heart valves, venous valves and arteries). A stent can be used to obtain and maintain the patency of the body passageway while maintaining the integrity of the passageway, and can be an alternative to surgery. Stent manufacture and usage are generally known in the medical arts.

One disadvantage of utilizing stents in a vessel is the potential development of a thrombis formation and/or cellular response within the stent causing a re-occlusion of the artery, the so-called neointimal hyperplasia. This may cause scar tissue (cell proliferation) to rapidly grow over or within the stent, or some other negative reaction. A common theory of re-occlusion of arteries is that development of a neointima is variable but can at times be so severe as to re-occlude the vessel lumen (i.e., restenosis), especially in the case of smaller diameter vessels, which often requires re-intervention. Another disadvantage of utilizing stents in a vessel is that the expansion of the vessel upon insertion of the stent can weaken the vessel and/or cause secretion of undesirable biological factors due to the stress exerted on the artery. There is an occasional tendency for clots to form at the site where a stent is implanted and it potentially damages a vessel wall. This tendency may be higher for drug-eluting stents. Since platelets are involved in the clotting process, subjects must take antiplatelet therapy (e.g., clopidogrel, aspirin) afterwards, usually for at least six months and perhaps indefinitely. But antiplatelet therapy may be insufficient to fully prevent clots; this and cell proliferation within, or near to, the stent may cause the conventional stents (e.g., “bare-metal” stents) or drug eluting stents to become blocked. Another disadvantage of utilizing stents in a vessel is biocompatibility responses to the foreign implant.

A drug-eluting stent is generally understood in the art to be a stent (i.e., a scaffold) placed into a vessel (e.g., a narrowed, diseased coronary artery) that slowly releases a drug, for example, to block cell proliferation. Blocking cell proliferation can prevent scar-tissue-like growth that, together with clots (i.e., thrombus), could otherwise block the stented vessel. For example, drug-eluting stents releasing an antiproliferative drug (drugs typically used against cancer or as immunosuppressants) can help avoid, at least in part, in-stent restenosis (re-narrowing or re-occlusion, either in part or in whole). Examples of current drug-eluting stents include Cypher™, a sirolimus-eluting stent (Cordis Corp., Johnson & Johnson) and Taxus™, a paclitaxel-eluting stent (Boston Scientific), both of stainless steel and using a polymer as a drug carrier. Other drugs reported to be used in conjunction with a stent include zotarolimus (ZoMaxx stent, Abbott Labs; Endeavor stent, Medtronic); everolimus (Champion stent, Xience stent, Abbott Labs). But recent studies have revealed that present drug eluting stents are associated with a 5 fold higher risk for thrombosis (with fatality results in one-third of patients who develop late thrombosis) compared to bare metal stents. Bavry et al. (2006) Am. J. Med. 119 (12), 1056-1061.

Current drug-eluting stents generally consist of three parts. The stent itself is an expandable framework, usually metal. Added to this is a drug, usually one to prevent the artery from being re-occluded, or clogged. These typically have been drugs already in use as anti-cancer drugs or drugs that suppress the immune system. Finally, there is a carrier which slowly releases the drug over months. The carrier is typically a polymer, although phosphorylcholine or ceramics have also been reported. Different carriers can release the loaded drug at different rates.

The stent is often delivered to the target area of the body passageway by a balloon and catheter system tracking over a guide wire. Once properly located, the balloon is expanded, plastically deforming the entire structure of the stent against the body passageway. Expansion can also crack and/or compress any plaque present in the vessel. The amount of force applied is usually at least that necessary to expand the stent (i.e., the force applied exceeds the minimum force above which the stent material will undergo plastic deformation) while maintaining the patency of the body passageway. At this point, the balloon is deflated and the balloon, catheter system, and guide wire are withdrawn from the lumen and subsequently removed from the body altogether. Ideally, the stent will remain in place and maintain the target area of the body passageway substantially free of blockage (or narrowing).

Progesterone (P4, or pregn-4-ene-3,20-dione) is a C-21 steroid hormone known to be involved in the female menstrual cycle, pregnancy (supports gestation) and embryogenesis of humans and other species. Progesterone belongs to a class of hormones called progestogens, and is the major naturally occurring human progestogen. Phylogenetic studies suggest that the estrogen receptor was the first to evolve as the target for the terminal hormone in the pathway for steroid biosynthesis; followed by the progesterone receptor (see Thornton (2001) Proc Natl Acad Sci 98, 5671-5676). Following a considerable sequence of evolutionary divergence additional receptors emerged that gave the intermediate compounds, androgens, glucocorticoids, and mineralocorticoids, novel signaling functions. (Thornton and Baker (2001) J Molec Endocrinol 26, 119-125). These intermediate compounds each act through their receptors to effect specific regulation of physiological activities important to homeostasis, reproduction, differentiation, development and immune response. These receptors are structurally distinct (see Goodman and Gillman's, The Pharmacological Basis of Therapeutics, 9^{th} Ed., McGraw-Hill Professional, 1995).

### SUMMARY OF THE INVENTION

The present invention is directed to compositions containing progesterone and their use in various formulations, medical device coatings, and methods of therapeutic treatment. The progesterone-containing formulations and medical device coatings described herein can maintain, or aid in maintaining, the opening of a body passageway. The progesterone-containing formulations and medical device coatings described herein can aid in reducing or eliminating undesirable cellular growth, such as smooth muscle cells or cancerous or pre-cancerous tissue, lesions, or cells.

In brief, the present invention provides progesterone-containing compositions, formulations, and/or medical device coatings to give functional properties such as, for example, vessel relaxative, anti-oxidative, anti-restenotic, anti-angiogenic, anti-thrombotic, anti-cancer, and/or anti-tumor effect. The progesterone-containing compositions, formulations, and/or medical devices described herein can, inter alia, minimize or eliminate inflammation, thrombosis, restenosis, neo-intimal hyperplasia, smooth muscle cell proliferation, rupturing of vulnerable plaque, dysplastic tissue, neoplastic progression, and/or other effects related to device implantation or treatment.

One aspect of the invention provides a drug eluting medical device. In some embodiments, the drug eluting device includes a medical device and a progesterone-containing composition. In some embodiments, the drug eluting device includes an eluting mechanism. In some embodiments, the eluting mechanism is a coating, reservoir, pore, duct, channel, chamber, side-port, or lumen. The eluting mechanism can be proximal to, distal to, lateral to, underneath, embedded within or on the device. The eluting mechanism can elute progesterone in vivo. In some embodiments, the drug eluting device includes at least one coating layer. In some embodiments, the drug eluting device includes a medical device comprising a drug-eluting mechanism (e.g., a well, pocket or crevice within the surface or body of a device) and a progesterone-containing composition. In some embodiments, the device includes both a coating layer and a drug-eluting mechanism. Usually, a coating layer is formed on at least a portion of a surface of the medical device and the coating layer will include the progesterone-containing composition. Such composition can be present in any of a number of drug eluting mechanisms, heretofore including a reservoir, pore, duct, channel, chamber, side-port, lumen, etc., within, proximal to, distal to, lateral to, underneath, embedded within or on the medical device. The progesterone-containing composition is usually present in a therapeutically effective amount. And the various components of the drug eluting medical device are configured such that the progesterone-containing composition is eluted from the medical device in vivo.

Another aspect of the invention provides for a method of treating a target tissue of a subject. The method generally includes providing a drug-eluting medical device; and introducing the drug eluting medical device to a target tissue of a subject in need thereof. The drug eluting medical device generally includes a medical device, a progesterone-containing composition, and at least one coating layer and/or drug eluting mechanism formed on at least a portion of a surface of the medical device. The coating layer(s) generally contains the progesterone-containing composition, and such composition is eluted from the medical device in vivo. According to the method, progesterone is eluted from the delivered medical device in a therapeutically effective amount.

Another aspect of the invention provides for an anti-angiogenic, anti-thrombotic, or anti-restenotic composition containing a therapeutically effective amount of progesterone and vitamin E. The therapeutically effective amount of progesterone in the composition is an amount that has an anti-angiogenic, anti-thrombotic, anti-neoplastic, and/or anti-restenotic effect in a subject.

Another aspect of the invention provides for an anti-angiogenic, anti-thrombotic, or anti-restenotic pharmaceutical formulation containing a therapeutically effective amount of progesterone and vitamin E along with a pharmaceutically acceptable carrier. The therapeutically effective amount of progesterone in the formulation is an amount that has an anti-angiogenic, anti-thrombotic, anti-neoplastic, and/or anti-restenotic effect in a subject.

Provided below are various embodiments of the different aspects of the invention described below. It is understood that reference to, for example, the progesterone-containing composition can include reference to such composition as occurring in the drug eluting medical devices, methods, compositions, or pharmaceutical formulations described herein. Likewise, reference to various components of the drug eluting medical device can include reference to such components as occurring in the drug eluting medical device or methods described herein.

In various embodiments, the progesterone-containing composition further comprises at least one additional therapeutic agent. For example, the additional therapeutic agent is an antiplatelet, anticoagulant, antifibrin, antiinflammatory, antithrombin, antiproliferative, antioxidants, and/or growth factors (e.g., VEGF). In various embodiments, the progesterone-containing composition further comprises vitamin E (alpha-tocopherol).

In various embodiments, the coating layer or drug eluting mechanism of the drug-eluting medical device is made up of, at least in part, a polymeric material. The drug eluting medical device can comprise a second coating layer or drug eluting mechanism, wherein the second coating layer or drug eluting mechanism comprises a polymeric material and the second coating layer or drug eluting mechanism acts as a barrier layer to further control elution of the progesterone-containing composition.

In various embodiments, the drug eluting medical device is a drug eluting stent. In various embodiments, the drug eluting medical device is configured to treat neointimal lesions; restenotic lesions; lesions within stents; vulnerable plaque lesions; bifurcated lesions or ostial lesions; or for use in coronary, cardiac, peripheral carotid, gastro-intestinal, gastro-esophageal, urologic, uterine, prostate, neurologic, vascular, organ, muscle, or body cavity applications. The drug eluting device can be configured to treat Barrett's esophagus. It can also be configured to treat dysplastic esophagus or non-dysplastic but diseased (e.g. cancerous or pre-cancerous) esophagus.

In various embodiments, the therapeutically effective amount has one or more effects such as an anti-angiogenic effect, anti-thrombotic effect, anti-restenotic effect, vessel-relaxative effect, anti-oxidative effect, anti-neoplastic, or combinations thereof.

### BRIEF DESCRIPTION OF THE DRAWINGS

The drawings, described below, are for illustrative purposes only and are not intended to limit the scope of the present teachings in any way.

FIG. 1 is a flow chart depicting, inter alia, suggested mechanisms underlying the effect of progesterone in anti-angiogenic, anti-restenotic, and/or anti-thrombotic applications.

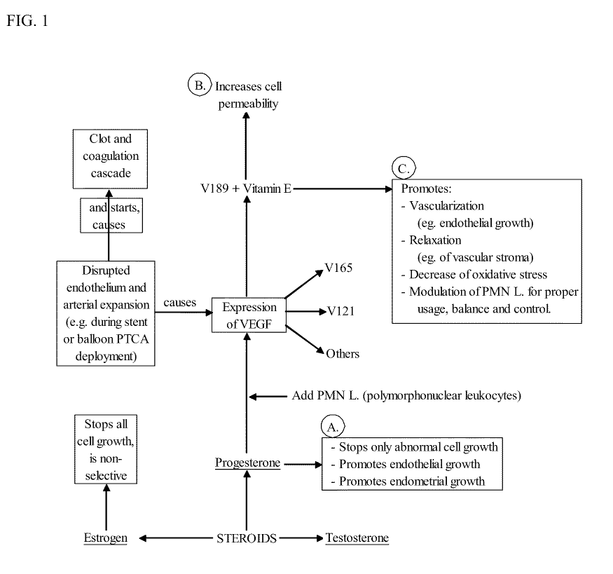

FIG. 2 is a line and scatter plot showing smooth muscle cell number as a function of time (days) under control and progesterone (10 μg/ml) treatment. Further details regarding methodology are available in Example 10.

FIG. 3 is a diagram depicting the structure of linoleic acid (C18:2c9c12) and two isomers of conjugated linoleic acid (C18:2c9t11 and C18:2t10c12).

FIG. 4 is a series of images of cell cultures. FIG. 4A shows control SMC at day 1. FIG. 4B shows progesterone treated SMC at day 1. Further details regarding methodology are available in Example 14.

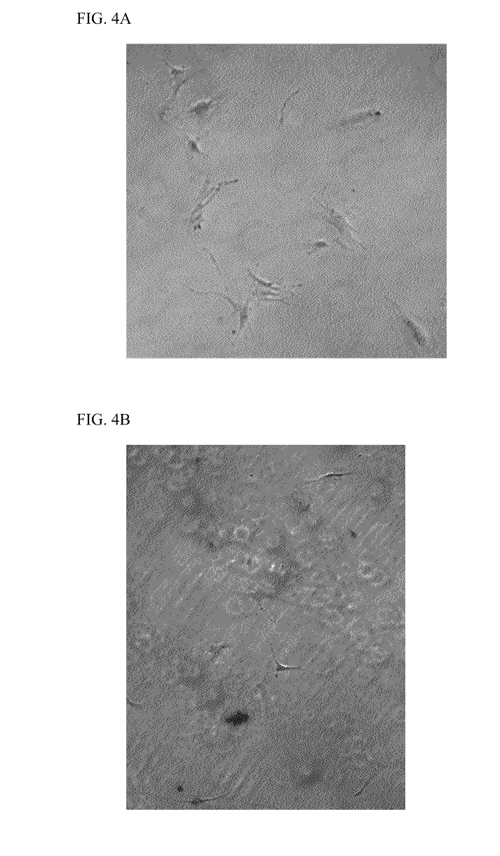

FIG. 5 is a series of images of cell cultures. FIG. 5A shows control EC at day 1. FIG. 5B shows progesterone treated EC at day 1. Further details regarding methodology are available in Example 14.

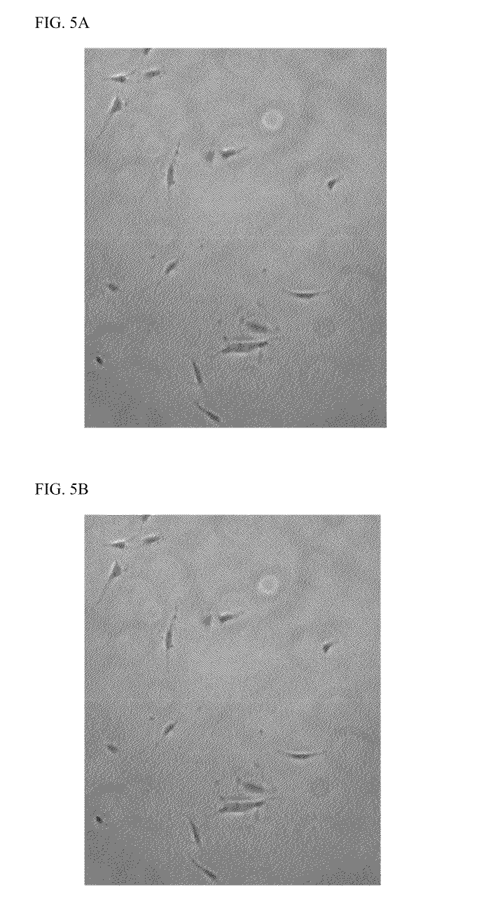

FIG. 6 is a line and scatter plot showing human coronary artery endothelial cell and aortic and coronary human smooth muscle cell percent change versus control over time. Progesterone is compared to control at each time point (Day's 1, 3, 6 and 8). The difference between progesterone and control has been plotted. Progesterone 10 and 30 mg decrease SMC's compared to control in a dose response fashion while progesterone 30 mg increases endothelial cells. Further details regarding methodology are available in Example 10 and Example 14.

FIG. 7 is a line and scatter plot showing human coronary endothelial cell and human coronary smooth muscle cell growth over time for control and treatment with 30 μg progesterone. Further details regarding methodology are available in Example 14.

### DETAILED DESCRIPTION OF THE INVENTION

The present invention relates to compositions and devices that can minimize or eliminate conditions and complications, such as inflammation, thrombosis, restenosis, neo-intimal hyperplasia, rupturing of vulnerable plaque, dysplasia, and/or other effects. More specifically, the present invention is directed to a progesterone-containing composition that can be administered directly and/or used in conjunction with a medical device to maintain opening of a body passageway. The progesterone-containing composition can also include one or more additional pharmacologically active therapeutic agents.

The progesterone-containing composition and devices can improve the results of bare metal, polymeric, bioresorbable, surface treated, combinations of these or any other non-progesterone containing devices, and allow constricted, blocked or diseased blood vessels, organs, tissues, or cells to remodel and/or heal. This may be in an open, relaxed position. Further, progesterone-containing compositions, applied directly (e.g., as in endoluminal paving or nanoparticles) or as a device coating, can reduce or eliminate restenosis, thrombosis, dysplasia, and/or inflammation associated with a diseased or pre-diseased part of the body, or when associated with implantation of a foreign device in a subject. Also, the progesterone-containing compositions and devices described herein can be combined with allogenic endothelial cells or non-allogenic endothelial cells, or cellular matrices with or without endothelial cells. Manufacturing various devices, such as stent systems, with the progesterone-containing composition described herein can impart many advantageous qualities to the resulting device systems.

Composition

The composition of the invention generally includes progesterone. Optionally, one or more additional active agents may be included in the progesterone-containing composition. Exemplary additional therapeutic agents include vitamin E (e.g., α-tocopherol) and/or conjugated linoleic acid. The progesterone-containing composition can be formulated for direct administration, device delivery, delayed delivery, time-released delivery, as a device coating, and/or a combination of one or more of these as described below.

Progesterone

Progesterone is a natural plant derived product, and also occurs naturally in the body. Progesterone belongs to a class of hormones called progestogens, and is the major naturally occurring human progestogen. Progesterone, like all other steroid hormones, is synthesized from pregnenolone, a derivative of cholesterol. Progesterone is involved in biosynthesis of, for example, the adrenal corticosteroids and sex hormones, including both estrogen and testosterone.

The progesterone-containing composition described herein can have the effect of minimizing or eliminating adverse events such as thrombosis, neo-intimal hyperplasia, restenosis, smooth muscle cell proliferation, inflammation, dysplasia, pre-dysplasia, and/or other deleterious effects. Such beneficial effects are provided in situ by coating a device, or delivery with a device, as described herein, so as to elute progesterone, and optionally additional agents, at a controlled rate over an extended period of time or as a single or multiple bolus. Progesterone can be used as the exclusive active ingredient in the composition or coated device, thereby avoiding deleterious side-effects associated with many currently employed drugs in coated stent applications. In contrast to current drugs employed in coated stent applications, progesterone is naturally occurring in the body and, as such, involves less deleterious side-effects. Alternatively, one or more additional active therapeutic agents can be included in the composition and/or coated device.

The progesterone-containing composition described herein can relax smooth muscle, including vascular smooth muscle cells; act as an anti-inflammatory agent; normalize, reduce, or prevent blood clotting; normalize vascular tone; regulate various types of collagen, which can aid in healing and strengthen blood vessels; eradicate or minimize dysplasic tissue (such as Barrett's esophagus) and/or decrease or eliminate the rate of neoplastic progression; and/or regulate deleterious effects of estrogen.

Anti-proliferation effects of the progesterone-eluting device can reduce or eliminate proliferation-associated conditions such as restenosis. Anti-inflammatory effects of the progesterone-eluting device can reduce or eliminate inflammatory complications associated with various diseases and disorders, such as inflammation associated with coronary heart disease. Progesterone can inhibit growth of smooth muscle cells, which have been shown to be involved in the restenotic process. It can also reduce or eliminate thrombis, clotting, and/or subsequent restenosis, due at least in part its ability to promote endothelial regeneration. The promotion of effective endothelial regeneration by the progesterone-containing composition can decrease the susceptibility of the treated vessel to late thrombosis, or thrombosis at any stage, in the healing process. Progesterone eluted from a coated device or delivered from a device described herein can also protect the integrity and function of cell membranes, thereby protecting against thrombosis, restenosis, and/or rupturing of vulnerable plaque. The various effects of progesterone described above can occur in a dose-dependent manner.

The progesterone-containing composition described herein can oppose various negative effects of estrogen. Estrogen is known to induce increased coagulability of blood and increase the risk of ischemic stroke. Thus, progesterone eluted from a coated device or delivered by a device can oppose the negative effects of estrogen, reducing potentially elevated blood coagulability and/or reducing the risk of ischemic stroke. Both elevated blood coagulability and the risk of ischemic stroke are understood to be related to clotting reactions in the body.

The progesterone-containing composition may contain progesterone or progesterone analogues that retain a substantial portion of the above described features. Other suitable progestogens may include, for example, allyloestrenol, dydrogesterone, lynestrenol, norgestrel, norethyndrel, norethisterone, norethisterone acetate, gestodene, levonorgestrel, medroxyprogesterone, and megestrol. Various synthetic progestins may not fulfill all or substantially all roles of progesterone, as many such synthetic progestins were designed solely to mimic progesterone's uterine effects. Preferably, the progesterone-containing composition and the coated device described herein contain natural progesterone, and not progestins (i.e., synthetically produced progestogens). Progesterone analogues, including synthetically produced progestogens, may be suitable provided they provide the desired reduction or elimination of conditions described above, such a restenosis, thrombosis, and/or inflammation.

The progesterone or progesterone analogue of the composition and coated device described herein can be in United States Pharmacoepia (USP) form, and preferably is in USP form in various embodiments. It is noted that most USP progesterone is extracted from plant sources, notably soy and yams. Soybeans contain the sterol stigmasterol, while yams contain the sterol diosgenin, both of which have progesterone-like effects. USP progesterone is generally produced by hydrolyzing extracts of soy or yam and converting saponins into sapogenins, from two of which, sarsasapogenin (soy) and diosgenin (yam), can be derived natural progesterone.

Progesterone for inclusion in the compositions described herein can be derived from a species of flowering plant Dioscorea. Preferably, progesterone for inclusion in the compositions described herein is derived from Dioscorea villosa, Dioscorea floribunda, Dioscorea macrostachya, and/or Dioscorea barbasco, and more preferably Dioscorea barbasco. The Mexican yam, Dioscorea barbasco, especially is known to have especially high levels of antioxidant effects, including cardiovascular protective and disease preventive effects. From a selected species, diosgenin (a type of saponin) from the yam can be derivatized to natural progesterone. In various embodiments, the plant source is selected at a stage (e.g., season, chronological age, developmental age, etc.) during which the compound of interest (e.g., diosgenin) is at its highest concentration within the tissues.

Progesterone, a steroid hormone, possesses a similar core structure as compared to female estrogenic hormones and male androgenic hormones, as well as cholesterol and adrenal steroid hormones. Where an implant device, cell wall, or implanted issue has progesterone embedded in its surface or structure, passing cholesterol in the blood may not be able to bind or embed itself into the implant device, cell wall, or tissue implant given the presence of progesterone occupying the adherence site. Furthermore, optional inclusion of vitamin E (i.e., a generic term for tocopherols and tocotrienols) in the composition may further repel cholesterol. Also, vitamin E may provide relaxative effects to the cell, tissue, vessel, and/or organ such that it can better accept the progesterone or progesterone and other therapeutic ingredient(s), and thus provide a better therapeutic and/or safer result. Optional inclusion of vitamin E may also be helpful in improving effectiveness, transport, and longevity of the progesterone, as well as providing anti-oxidative benefits to the vessel.

The progesterone compositions described herein may help to attract and increase concentration of High Density Lipoprotein (HDL). High concentrations of HDL (over 60 mg/dL) have been shown in epidemiological studies to have protective value against cardiovascular diseases such as myocardial infarction and ischemic stroke. Low concentrations of HDL (below 40 mg/dL for men, below 50 mg/dL for women) are a positive risk factor for these atherosclerotic diseases. In contrast, the progesterone compositions described herein may help to repel and/or decrease concentration of Low Density Lipoprotein (LDL).

While being under no obligation to provide a mechanism, nor limiting the present invention in any way by providing such, potential mechanisms for the progesterone-containing composition include, but are not limited to inhibition of nuclear transcription factors, modulation of growth factor activity or receptor binding, regulation of extracellular matrix production, direct inhibition of smooth muscle cell proliferation and migration, and/or anti-inflammatory effect. For example, progesterone selectively increases V189 (also known as VEGF189, an isoform of vascular endothelial growth factor, VEGF) expression in perivascular decidual endometrial cells during the mid-late secretory phase of the menstrual cycle, and during early gestation, where V189 increases capillary permeability, similarly to other VEGF isoforms (Ancelin et al. (2002) Proc Natl Acad Sci USA 99, 6023-6028). Capillary permeability may be helpful in promoting endothelialization, thus providing a positive foundation for a successful stent implantation, medical device implant, or medical device usage in the human body. In contrast to progesterone, estrogens are not selective, or not as selective, and may increase expression of all VEGF isoforms (see Ancellin et al. (2002)). It is progesterone's ability to selectively induce V189 that may, at least in part, contribute to the efficacy of the progesterone-containing compositions described herein.

Further potential mechanisms for the progesterone-containing compositions described herein are provided, but provision of such is understood to not limit the scope of the invention in any way. The human endometrium is an accepted model for the study of physiological angiogenes, given that it is a tissue that undergoes rapid cyclic changes under the control of ovarian hormones, estradiol and progesterone. Polymorphonuclear leukocytes (PMN) in intimate contact with endometrial endothelium have been shown to be a source of intravascular VEGF for vessels undergoing angiogenesis (Ancelin et al. (2002)). While PMN are found in only small numbers in intact tissue, elevated levels of PMN are found in areas of tissue breakdown (e.g., in the human endometrium during the premenstrual and menstrual periods). PMN and NK cells (CD 56+) also infiltrate the endometrial stroma during the luteal phase and pregnancy, under the influence of progesterone.

It is thought that individual VEGF isoforms may have different functions on different aspects of vascular growth (Herve et al. (2005) Experimental Cell Research 309, 24-31). For example, VEGF is up-regulated by the myocardial ischemia that develops as a result of epicardial coronary obstruction (Cheng et al. (1997) Proc Natl Acad Sci USA 94(22), 12081-12087). But some isoforms of VEGF have been shown to mediate various deleterious effects. It has been shown that the V189 isoform of VEGF induces PMN chemotaxis, probably by binding to the Flt-1 receptor, and that VEGF-induced PMN migration is involved in angiogenesis and/or inflammation, via an outcome regulatory loop (Ancelin et al. (2002)). V189 has also been shown to up-regulate expression of Flk-1/KDR and stimulates endothelial cell migration (Herve et al. (2005) Experimental Cell Research 309, 24-31). The Flt-1 and Flk-1/KDR receptors are understood to mediate the angiogenic effects of VEGF (Herve et al. (2006) Journal of Endocrinology 188, 91-99). Progesterone or progesterone with vitamin E may have a chemotaxis effect on neutrophils (e.g., PMN) via relationship with VEGF189. It has also been shown that V189-induced PMN migration on fibronectin is dependent on B1-integrin (Ancelin et al. (2002)). Further, V189 has been shown to induce cell proliferation on corneal endothelial cells (Jonca et al. (1997) J Biol. Chem. 272(39), 24203-9). Also, V189 over-expression enhanced angiogenicity in mice but with reduced tumorigenicity, hemorrhaging, and rupturing observed with over-expression of other VEGF isoforms (Cheng et al. (1997) Proc Natl Acad Sci USA 94(22), 12081-12087). Such reduction of hemorrhaging and rupturing may have beneficial implications for the reduction in thrombosis. It is known, for example, that smooth muscle cells have progesterone receptors mediating endometrial angiogenesis (Perrot-Applanat et al. (2000) Steroids 65(10-11), 599-603). So, V189 may seal off and prevent continued tumor cell proliferation, and also prevent or reduce vascular smooth muscle cell proliferation. Because individual VEGF isoforms may have different functions on different aspects of vascular growth as explained above, V189 may play a role in balancing endothelial proliferation and the prevention or minimization of restenosis, especially in the presence of the progesterone compound as described herein. Specifically, V189 may inhibit smooth muscle cell proliferation and promote endothelialization.

Again, progesterone has been shown to selectively increase V189 (isoform of VEGF) expression. Thus, VEGF, V189 isoform, Flt-1 and Flk-1/KDR receptors, PMN, and B1-integrin-fibronectin interactions may be involved in the cascade of lesion disease. And through selectively increasing expression of V189 and mediating the effects of Flt-1 and/or Flk-1/KDR receptors, the progesterone-containing compositions described herein may promote endothelialization, prevent restenotic lesions from forming, and/or prevent clots and/or thrombosis from occurring at the site of a newly deployed drug-eluting stent or medical device.

A progesterone eluting stent provides for local pharmacodynamic activity to attenuate a natural inflammatory vascular response to an injury caused by an interventional coronary procedure, such as stenting. For example, a total drug exposure from an about 15 mm coronary drug eluting stent loaded with about 300 μg (0.3 mg) of progesterone, eluting over about 1 to about 3 months, represents a fraction of the 1-5 mg daily de novo biosynthesis of man or systemic intravenous doses (>70 mg in 3 days) currently under evaluation in the clinical environment.

The calculation of dosages, dosage rates and appropriate duration of treatment with the progesterone-containing composition and/or coated device are within the ordinary skill of the art. Furthermore, additional therapeutic agents can be loaded at desired concentration levels per methods well known in the art to render the device ready for implantation.

Vitamin E

The progesterone-containing composition, coating, and/or device can further contain Vitamin E. Vitamin E can increase effectiveness of the progesterone-containing composition for direct delivery and/or when coated on or in a device. Similarly, vitamin E can be used in conjunction with the progesterone-containing composition for prevention and/or treatment of other disorders related to uncontrolled cell growth, such as cancerous or pre-cancerous conditions.

Vitamin E is a generic term for tocopherols (alpha, beta, gamma, delta) and tocotrienols (alpha, beta, gamma, delta), which are fat-soluble antioxidant compounds that can stop production of reactive oxygen species and are known to protect cell membranes, active enzyme sites, and DNA from free radical damage. Of the Vitamin E compounds, α-tocopherol has the highest bioavailability.

Vitamin E can be included in the composition, device-coating, or delivery device described herein in a variety of forms, including any or all of the eight different natural isomers (four tocopherols and four tocotrienols) and each of their alpha, beta, gamma, and delta forms. The alpha, beta, gamma, and delta forms are variable on the number of methyl groups on the chromanol ring of vitamin E. For example, the vitamin E in the progesterone-containing composition or coated device can be E307 α-tocopherol), E308 (γ-tocopherol), and E309 (δ-tocopherol). Preferably, the progesterone-containing composition and/or device coating contains a tocopherol, such as α-tocopherol. The Vitamin E of the composition, device-coating, or delivery device described herein can be a natural form, synthesized form, or combination of these.

The progesterone-containing composition and/or device coating can contain fully naturally occurring vitamin E, natural mixed tocopherols (e.g., mixed tocopherols with an additional 25%-200% w/w d-beta-, d-gamma-, and d-delta-tocopherol), high gamma-tocopherol fractions, semi-synthetic vitamin E esters (e.g., d-alpha tocopheryl ester (acetate or succinate)), synthetic vitamin E (e.g., d, 1-tocopherol or d, 1-tocopheryl acetate), or combinations thereof. Naturally occurring α-tocopherol is traditionally recognized as the most active form of vitamin E in humans. Preferably, the α-tocopherol form and/or the mixed tocopherol form of vitamin E is included in the progesterone-containing composition or coated device. Vitamin E contained in the progesterone-containing composition or device coating can be mycellized vitamin E.

Vitamin E, as contained in the progesterone-containing composition or device coating can, among other effects, act as an anticoagulant; improve or facilitate delivery; prevent the formation of blood clots; facilitate penetration of biological membranes, cells, tissues, vessels, and/or organs; prevent oxidative stress; act as a negatively charged component; provide relaxative effects; and/or limit oxidation of LDL-cholesterol. The anticoagulant properties of vitamin E, along with its ability to prevent formation of blood clots, can serve to reduce or eliminate clot-related complications such as thrombosis. Prevention of oxidative stress can reduce the level of trauma to the target tissue (e.g., vessel) during and after implantation of, or treatment with, a device. Limiting oxidation of LDL-cholesterol can reduce blockages and/or re-occlusions in coronary arteries that may lead to atherosclerosis, stroke, and/or heart attacks. Alpha tocopherol is a major antioxidant in LDL, where it reduces LDL oxidation; and one LDL particle contains about six molecules of alpha tocopherol. Vitamin E depletion in LDL may trigger LDL oxidation; and the addition of micromolar concentrations of vitamin E can inhibit LDL oxidation (Nakamura et al. (2008) Nutrition and Metabolism 5, 22). The ability of vitamin E to increase penetration of biological membranes can act as a carrier for progesterone and/or other therapeutic agents of the composition or coated device.

Epidemiological and clinical studies indicate that vitamin E may reduce the risk of cardiovascular disease (CVD). Modulation of adhesion molecule expression and chemokine production by vitamin E may contribute to its beneficial effect. Enrichment of confluent human aortic endothelial cells (HAEC) or U937 monocytic cells with increasing doses of vitamin E (d-alpha-tocopherol, 20, 40, and 60 micromol/1 for 20 h) inhibited their adhesion when either or both cell types were stimulated with interleukin (IL)-1beta (Wu et al. (1999) Atherosclerosis 147(2), 297-307). Enrichment of HAEC with the same doses of vitamin E suppressed IL-1beta-stimulated expression of intercellular adhesion molecule-1 (ICAM-1), vascular cell adhesion molecule-1 (VCAM-1), and endothelial leukocyte adhesion molecule-1 (E-selectin) (Wu et al. (1999)). Supplementation with increasing doses of vitamin E up to 60 micromol/1 was not effective in preventing spontaneous production of monocyte chemoattractant protein-1 (MCP-1), but supplementation with vitamin E at 60 micromol/1 reduced IL-8 production significantly (Wu et al. (1999)). However, IL-1beta-induced productions of both MCP-1 and IL-8 were dose-dependently suppressed by enrichment of cells with vitamin E. Vitamin E, at the doses used, did not significantly change the spontaneous production but dose-dependently inhibited the IL-1beta-induced production of inflammatory cytokine IL-6 (Wu et al. (1999)). Thus, vitamin E may inhibit production of chemokines and inflammatory cytokines, in addition to inhibiting adhesion of HAEC to monocytes by reducing expression of adhesion molecules when cells were activated with an inflammatory cytokine. These mediators are actively involved in the pathogenesis of atherosclerosis. Therefore, their inhibition by vitamin E may contribute to vitamin E's reported reduction in risk of CVD (see Wu et al. (1999).

Vitamin E, when included in the progesterone-containing composition or device coating, can have a relaxative effect. Such effect can allow constricted, closed, or clogged blood vessels to open, become less restricted, and/or be easier to treat. Because an interventional and/or intrusive device can be traumatic to the vessel, vitamin E delivered to the vessel before, during, and/or or after delivery, deployment, and/or expansion can result in reduction of thrombosis, restenosis, inflammation, and/or other adverse events. Vitamin E can aid in the reduction of fibrous tumors in, on, or near the areas of administration. Vitamin E can control blood lipoperoxidation and maintain antioxidant status.

Where used in conjunction with (e.g., before, during, after, or formulated with) progesterone, vitamin E can reduce oxidative stress and aid progesterone migration in the areas within the membrane, tissue, and/or cellular environment needing its benefit. Vitamin E can aid dissolution/formulation of progesterone and increase absorption of the composition into the lymphatic system. The vitamin E, when used in conjunction with progesterone, can increase oxygenation in the tissues near the area of administration. Progesterone and vitamin E can improve the electrical environment of the coated stent or device, promote endothelialization, and prevent or inhibit smooth muscle cell proliferation.

The calculation of dosages, dosage rates, and appropriate duration of treatment as related to the vitamin E content of the composition and/or device coating are within the ordinary skill of the art. Exemplary ratios of progesterone to vitamin E in the compositions described herein can be from about 1:100 to about 100:1, preferably about 1:10 to about 10:1 (e.g., about 1:9, 1:8, 1:7, 1:6, 1:5, 1:4, 1:3, 1:2, 1:1, 2:1, 3:1, 4:1, 5:1, 6:1, 7:1, 8:1, or 9:1), and more preferably about 3:1. An exemplary effective amount of vitamin E in or on a composition, coating or device as described herein can range between about 10 μg and about 1000 μg. For example, an effective amount of vitamin E can range between about 250 μg and about 750 μg. As another example, an effective amount of vitamin E can be about 300 μg. In some embodiments, a composition, coating or device can be configured to elute Vitamin E at a rate of about 1 μg to about 100 μg per day. For example, a composition, coating or device can be configured to elute Vitamin E at a rate of about 10 μg per day. As another example, a stent loaded with 300 μg and eluting at 10 μg per day would provide treatment for 30 days.

Conjugated Linoleic Acid

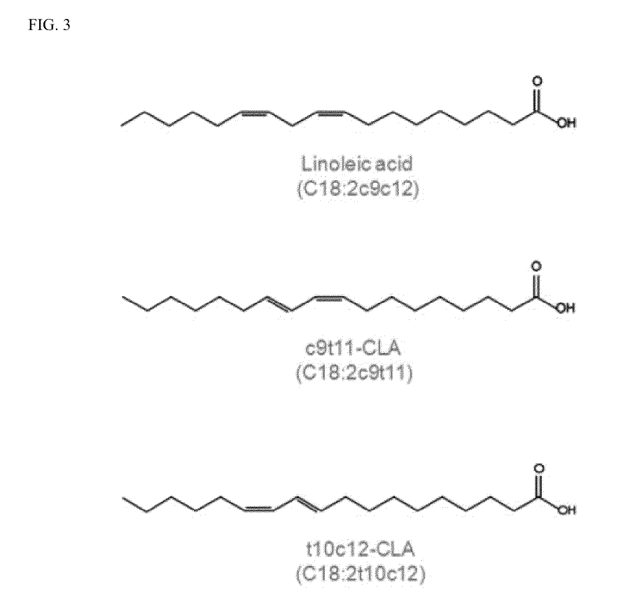

The progesterone-containing composition, coating, and/or device can further contain conjugated linoleic acid. Conjugated linoleic acids (CLAs) are a group of isomers of linoleic acid (LA) (e.g., C18:2c9c12) characterized by the presence of conjugated double bonds (see e.g., FIG. 3 depicting CLAs C18:2c9t11 and C18:2t10c12). Exemplary CLAs include, but are not limited to, C18:2 linoleic acid variants such as 9,11-CLA (i.e., 9,11-octadecadienoic acid) and 10,12-CLA (i.e., 10,12-octadecadienoic acid). In some embodiments, the CLA is selected from one or more of cis-9,trans-11 CLA (i.e., rumenic acid); trans-9,cis-11 CLA; cis-9,cis-11 CLA; trans-9,trans-11 CLA; cis-10,cis-12 CLA; trans-10,cis-12 CLA; cis-10,trans-12 CLA; and trans-10,trans-12 CLA.

Conjugated linoleic acids (CLAs) are biologically highly active lipid compounds that can inhibit atherosclerotic plaque development and/or regress pre-established atherosclerotic plaques. Anti-atherogenic effects of CLAs in vivo may derive, at least in part, from inhibition of inflammatory and vasoactive mediator release from endothelial cells (ECs) and smooth muscle cells (SMCs). Given that significant levels of CLA metabolites, reported to have significant biological activities, are detectable in ECs and SMCs, anti-atherogenic effects observed with CLAs may be mediated not only by CLAs but also by their metabolites (see Eder and Ringseis (2010) Mol. Nutr. Food Res. 54, 17-36).

CLAs may act as ligands and activators of peroxisome proliferator-activated receptors (PPARs) (Yu et al. (2002) Biochim. Biophys. Acta 1581, 89-99), which are known to attenuate pro-atherogenic events by inhibiting pro-inflammatory gene expression (see Duval et al. (2002) Trends. Mol. Med. 8, 422-430).

CLAs may possess anti-thrombotic properties, based on the observation that CLA isomers (e.g., C18:2c9t11, C18:2t10c12, and C18:2t9t11) and CLA mixtures can inhibit platelet aggregation in in vitro aggregation experiments performed with either platelet suspensions or whole blood (see Li et al. (2006) Eur. J. Pharmacol. 545, 93-99). An important function of the endothelium is to maintain an anti-thrombogenic blood-tissue interface by regulating the secretion of hemostatic (e.g., tissue factor (TF), PAF, plasminogen activator inhibitor-1) and fibrinolytic (e.g., tissue-plasminogen activator, thrombomodulin) factors. Because CLA isomers (e.g., C18:2c9t11, C18:2t10c12) and a CLA isomeric mix can inhibit EC production of PAF, which has stimulatory effects on platelet activation, suggests that CLAs may exhibit anti-thrombotic effects by modulating EC function (see Sneddon et al. (2006) Biochim. Biophys. Acta 1761, 793-801).

CLAs (isomers and mixtures) have an inhibitory effect on eicosanoid production. In vascular SMCs, CLA isomers (e.g., C18:2c9t11, C18:2t10c12) can attenuate secretion of eicosanoids (e.g., PGI2 and PGE2), like in ECs. (see Ringseis et al. (2006) Biochim. Biophys. Acta 1760, 290-300; Ringseis et al. (2006) Int. J. Vit. Nutr. Res. 76, 281-289).

Additional Therapeutic Agents

Additional therapeutic agents can be included in the progesterone-containing composition. For example, the composition can include one or more additional therapeutic agent(s) that can inhibit the activity of vascular smooth muscle cells (e.g., inhibiting abnormal or inappropriate migration and/or proliferation of smooth muscle cells for the inhibition of restenosis). As another example, the composition can include one or more additional therapeutic agent(s) capable of exerting a therapeutic or prophylactic effect for a diseased condition (e.g., enhancing wound healing in a vascular site or improving the structural and elastic properties of the vascular site). As another example, the composition can include one or more additional therapeutic agent(s) capable of exerting a therapeutic or prophylactic effect for a diseased or pre-diseased condition (e.g., eradicating or minimizing dysplastic tissue and/or decreasing the rate of neoplastic progression, such as in Barrett's esophagus).

The additional therapeutic agent(s) can include antiproliferative, antineoplastic, anti-inflammatory, antiplatelet, anticoagulant, antifibrin, antithrombin, antimitotic, antibiotic, antiallergic, antioxidant substances, vascular cell growth modulators, and/or vascular cell growth factors. Examples of such antiproliferative substances include actinomycin D, or derivatives and analogs thereof (Sigma-Aldrich, Inc., WI; COSMEGEN, Merck & Co., N.J.). Examples of such antineoplastics and/or antimitotics include paclitaxel (e.g., Taxol™, Bristol-Myers Squibb Co., CT), docetaxel (e.g., Taxotere™, Aventis S.A., Germany), methotrexate, azathioprine, vincristine, vinblastine, fluorouracil, doxorubicin hydrochloride (e.g., Adriamycin™, Pharmacia & Upjohn, N.J.), and mitomycin (e.g., Mutamycin™, Bristol-Myers Squibb Co.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax a (Biogen, Inc., MA). Examples of such cytostatic or antiproliferative agents include angiopeptin, angiotensin converting enzyme inhibitors such as captopril (e.g. Capoten™ and Capozide™, Bristol-Myers Squibb Co.), cilazapril or lisinopril (e.g., Prinivil™ and Prinzide™ from Merck & Co., Inc.); calcium channel blockers (such as nifedipine), colchicine, fibroblast growth factor (FGF) antagonists, fish oil (omega 3-fatty acid), various forms of omega 3, omega-6 and/or omega-9 fatty acids, conjugated linoleic acid, conjugated linoleic acid isomers (such as C18:2c9t11 and C18:2t10c12), histamine antagonists, lovastatin (an inhibitor of HMG-CoA reductase, a cholesterol lowering drug, brand name Mevacor™, Merck & Co., Inc.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents which may be appropriate include alpha-interferon, omega-interferon, modified or genetically engineered epithelial cells, rapamycin and its derivatives and analogs, and dexamethasone.

While the foregoing additional therapeutic agents have been used to prevent or treat restenosis, they are provided by way of example and are not meant to be limiting, since other therapeutic drugs may be known or developed which are equally applicable for use with the progesterone-containing composition described herein. The treatment of diseases using the above therapeutic agents is known in the art. The calculation of dosages, dosage rates and appropriate duration of treatment are likewise within the ordinary skill of the art. Furthermore, additional therapeutic agents can be loaded and/or coated at desired concentration levels per methods well known in the art to render a device ready for implantation.

As an example, heparin can be included in the progesterone-containing composition delivered at or around the time of device implantation (i.e., before, during, and/or after) and/or coated on or in the device. Heparin is a potent anticoagulant and is known to inhibit neointimal hyperplasia after balloon injury or implantation of a stent (see e.g., Frederick et al. (2001) Circulation 18(25), 3121-3124).

It is also contemplated that the progesterone-containing compositions described herein can be co-administered, or co-formulated with other agents, such as micro-organisms (e.g., alive, dead, attenuated), enzymes, coenzymes, ferments, fermentates, antigens, antibodies, harvested tissue, etc.

The various agents described herein, including progesterone and/or vitamin E, can be further derivatized by, for example, attachment of a DNA, nucleotide, nucleoside, sugar, starch, tannin, saccharide, polysaccharide, cellulose, glycoside, vitamin, etc. For example an agent could be attached (bonded, chelated, complexed) to a carbohydrate compound which is a saccharide and whose monomeric units are polyhydroxy mono-aldehydes or polyhydroxy mono-ketones, having the formula CₙH₂Oₙ, wherein n is five or six, or the corresponding cyclic hemiacetals thereof, or the reaction derivatives thereof in which the carbon skeleton and the carbonyl function or hemiacetal function of the saccharide unit are not destroyed; and the derivatives thereof.

Composition Formulation

The progesterone-containing compositions described herein can be formulated by any conventional manner using one or more pharmaceutically acceptable carriers and/or excipients as described in, for example, Gennaro (2005) Remington: The Science And Practice Of Pharmacy, 21st ed., Lippincott Williams and Wilkins, ISBN-10: 0781763789; Rowe et al. (2005) Handbook of Pharmaceutical Excipients, 5th ed., APhA Publications, ISBN-10: 1582120587; Brunton et al. (2005) Goodman & Gilman's The Pharmacological Basis of Therapeutics, 11th ed., McGraw-Hill Professional, ISBN-10: 0071422803; and Gibson (2001) Pharmaceutical Preformulation and Formulation: A Practical Guide from Candidate Drug Selection to Commercial Dosage Form, Informa Healthcare, ISBN-10: 1574911201, incorporated herein by reference in its entirety.

Such formulations can contain a therapeutically effective amount of the active agent(s), preferably in purified form (e.g. USP grade of progesterone), together with a suitable amount of carrier so as to provide the form for proper administration to a subject. As recognized in the art, the pharmaceutical formulation (comprising progesterone and, optionally, vitamin E or conjugated linoleic acid) can include, for example, a carrier, solvent, adjuvant, emulsifier, wetting agent, solubilizer, surface active agent, extending agent, buffering agent, etc. The formulation should suit the mode of administration. The progesterone-containing compositions can be formulated by known methods for administration to a subject using several routes which include, but are not limited to, parenteral, pulmonary, oral, topical, intradermal, intramuscular, intraperitoneal, intravenous, subcutaneous, intracutaneous, intrasternal, intraarticular, intrathecal, intranasal, epidural, endothelial, endometrial, endoluminal, ophthalmic, buccal, transmural, vaginal, penile, and rectal. Progesterone can also be administered in combination with one or more additional agents and/or together with other biologically active or biologically inert agents. Such biologically active or inert agents may be in fluid, chemical, vapor, plasma, or mechanical communication with progesterone and or other agent(s) or attached to progesterone and or other agent(s) by ionic, covalent, Van der Waals, hydrophobic, hydrophilic or other physical forces. The progesterone-containing compositions described herein can be lyophilized where appropriate for formulation and administration route.

A therapeutically effective amount of one of the agents described herein can be employed in pure form or, where such forms exist, in pharmaceutically acceptable salt form and with or without a pharmaceutically acceptable excipient. For example, the agents of the invention can be administered, at a reasonable benefit/risk ratio applicable to any medical treatment, in an amount sufficient to minimize or eliminate inflammation, thrombosis, restenosis, neo-intimal hyperplasia, rupturing or progression of vulnerable plaque, dysplastic tissue growth, neoplastic progression, and/or other related effects, or to promote endothelial regeneration.

Toxicity and therapeutic efficacy of such agents can be determined by standard pharmaceutical procedures in cell cultures and/or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The amount of an agent that may be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses. Agent administration can occur as a single event or over a time course of treatment. For example, an agent can be administered by a fraction of a second, by the second, by the minute, hourly, daily, weekly, bi-weekly, monthly, or yearly. For some conditions, treatment could extend from several hours to several days to several weeks to several months or even a year or more. In some embodiments, the therapeutically effective amount can be delivered from a drug eluting stent, osmotic pump, or other medical device, over the course of 30, 45, or 90 days, in an amount effective to inhibit smooth muscle cell proliferation while promoting regeneration of the endothelial lining. Such effects may reduce or eliminate the need for dual anti-platelet therapy (DAPT).

The specific therapeutically effective dose level for any particular subject will depend upon a variety of factors including the disorder being treated and the severity of the disorder; activity of the specific agent employed; the specific composition employed; the age, body weight, general health, sex and diet of the patient; the time of administration; the route of administration; the rate of excretion of the specific agent employed; the duration of the treatment; drugs used in combination or coincidental with the specific agent employed and like factors well known in the medical arts. It will be understood by a skilled practitioner that the total hourly, daily, weekly, or monthly usage of the agents for use in the present invention will be decided by the attending physician within the scope of sound medical judgment. In addition, various dosage formulations can be provided in a packaged product that is made available to the treating physician. For example, different formulations and/or dosages can be provided in the same package. It is within the skill of the art for a treating physician to determine which formulation and/or dosage is most appropriate for the given condition and/or subject.

The progesterone-containing compositions described herein can be micronized so as to enhance the rate of absorption and hence the effective level in the body. The progesterone-containing compositions described herein can be compounded in an oil base, extending effectiveness in the cardiovasculature, peripheral anatomy, neurovasculature, gastro-intestinal, gastro-esophageal, vaginal, prostrate, and elsewhere in the body. Because an oil base is absorbed through the lymphatic system first, the progesterone-containing composition can be screened from enzymes in the wall of the intestine or in the liver, and allow several passes through the body before being cleared via the liver. Preferably, the progesterone-containing composition is formulated, at least in part, in oils comprising long-chain fatty acids

Controlled-release (or sustained-release) preparations can be formulated to extend the activity of the agent and reduce dosage frequency. Controlled-release preparations can also be used to effect the time of onset of action or other characteristics, such as blood levels of the agent, and consequently affect the occurrence of side effects.

Controlled-release preparations can be designed to initially release an amount of an agent that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized and/or excreted from the body. The controlled-release of an agent may be stimulated by various inducers, e.g., change in pH, change in temperature (e.g., cryotherapy), enzymes, water, density, salt concentration, a light source (e.g., ultraviolet light), a radiofrequency, a radiation source (e.g., gamma, infrared, or x-ray), magnetic resonance, magnetic signal, electrical impulse, sound wave (e.g., ultrasound), or other physiological conditions or molecules. For example, the controlled release system can be a gas filled liposphere, activated by time, heat, cold, energy, ultrasound, any of the methods listed above, or other energy source.

Controlled-release systems may include, for example, an infusion pump (or infusion-like pump) that may be used to administer the agent in a manner similar to that used for delivering insulin or chemotherapy to specific organs or tumors. Typically, using such a system, the agent is administered in combination with a biodegradable, bioresorbable, bioerodable, and/or biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

The agents of the invention may be administered by other controlled-release means or delivery devices that are well known to those of ordinary skill in the art. These include, for example, hydropropylmethyl cellulose, other polymer matrices, polymer delivery molecules, gels, permeable membranes, osmotic systems, multilayer coatings, microparticles, nanoscaffolds, nanofibers, nanogels, nanoparticles, polymersome, polymer micelles, liposomes, microspheres, or the like, or a combination of any of the above to provide the desired release profile in varying proportions. Other methods of controlled-release delivery of agents will be known to the skilled artisan and are within the scope of the invention.

The progesterone-containing compositions described herein can be administered through a variety of routes well known in the arts. Examples include, but are not limited to, direct injection (e.g., systemic or stereotactic), transmural, oral delivery, inhalation delivery, minimally invasive delivery (e.g., as in minimally invasive CABG procedures that go through the rib cage), pulmonary delivery, implantation of cells engineered to secrete the factor of interest, drug-releasing biomaterials, implantable matrix devices, implantable pumps, injectable gels and hydrogels, liposomes, micelles (e.g., up to 30 μm), nanospheres (e.g., less than 1 μm), microspheres (e.g., 1-100 μm), reservoir devices, etc.

The progesterone-containing compositions described herein can be encapsulated and administered in a variety of carrier delivery systems. Examples of carrier delivery systems include microspheres (see generally, Varde & Pack (2004) Expert Opin. Biol. 4(1) 35-51), nanospheres (see generally, Mu et al. (2002) Journal of Controlled Release 80, 129-144; Mozafari (2007) Nanomaterials and Nanosystems for Biomedical Applications, Springer, ISBN-10: 1402062885), nanogels (see generally Arayne et al. (2007) Pak J Pharm Sci 20(4), 340-348), hydrogels (see generally, Sakiyama et al. (2001) FASEB J. 15, 1300-1302), polymeric implants (see generally, Teng et al (2002) Proc. Natl. Acad. Sci. U.S.A. 99, 3024-3029), smart polymeric carriers (see generally, Stayton et al. (2005) Orthod Craniofacial Res 8, 219-225; Wu et al. (2005) Nature Biotech (2005) 23(9), 1137-1146), and liposomes (see generally Galovic et al. (2002) Eur. J. Pharm. Sci. 15, 441-448; Wagner et al. (2002) J. Liposome Res. 12, 259-270). The carrier delivery system can incorporate a targeting ligand, such as an antibody (e.g., monoclonal antibody, antibody fragment, antibody-based fusion molecule, etc) specific for target cells/tissue (see generally, Radbruch et al. (2007) Immunotherapy in 2020: Visions and Trends for Targeting Inflammatory Disease, Springer, ISBN-10: 3540708502).

Carrier-based systems for use in various embodiments described herein can: provide for intracellular delivery; tailor agent release rates; increase the proportion of agent that reaches its site of action; improve the transport of the agent to its site of action; allow co-localized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; and/or improve shelf life of the product.

In various embodiments, the progesterone-containing compositions described herein, optionally including vitamin E or conjugated linoleic acid, can be delivered via liposome. As an example, the liposome delivery system can have a particle size of about 100 nm to about 300 nm, preferably about 180 nm to about 235 nm, and most preferably about 200 nm. Liposome of such sizes have been shown to increase the efficiency of delivering steroidal compositions to atherosclerotic lesions, through enhanced uptake by macrophages and foam cells in the lesions, while minimizing complications (Chono et al. (2005) Journal of Drug Targeting 13(4) 267-276).

In various embodiments, vitamin E can be used as an emulsifier in the preparation of progesterone-containing compositions in nanosphere delivery systems. As an emulsifier, vitamin E can, at least in part, stabilize the dispersed-phase droplets formed during emulsification, inhibit coalescence of droplets and determine the particle size, size distribution, the morphological properties and the release property of the nanospheres. Furthermore, natural surfactants such as vitamin E can have fewer side effects and better performance in preparation of polymeric nanospheres for clinical administration (e.g., anti-restenotic, anti-neoplastic, and/or anti-thrombotic) of the compositions described herein. Similarly, progesterone, via its structural similarity to cholesterol, can likewise act as a natural emulsifier in the preparation of polymeric nanospheres. Nanosphere and nanoparticulate delivery systems can improve bioavailability of the progesterone-containing compositions described herein by, for example, improving drug diffusion through biological barriers, permeation of cells for cellular internalization, permeation of connective tissue, and reducing capillary clogging. Nanosphere and nanoparticulate can include gelatin and albumin nanoparticles and magnetic nanoparticles. Nanosphere and nanoparticulate can incorporate targeting ligands for directed delivery of the progesterone-containing compositions described herein (see e.g., Arayne et al. (2007) Pak J Pharm Sci 20(4), 340-348). For example, the progesterone-containing compositions and coated devices described herein can be encapsulated in, and delivered by, fibrin targeted, lipid encapsulated, liquid perfluorocarbon nanoparticles (Arayne et al. (2007)). As another example, targeted delivery can utilize adhesion molecules such as vascular cell adhesion molecule-1 (VCAM) as a targeting ligand (Arayne et al. (2007)).

Various other delivery systems are known in the art and can be used to administer the agents of the invention. Moreover, these and other delivery systems may be combined and/or modified to optimize the administration of the agents of the present invention.

Coated Devices

Progesterone-containing compositions described herein, and formulations thereof, can be used to coat the surface of a variety of implantable devices, for example: drug-delivering vascular stents (e.g., self-expanding stents typically made from nitinol, balloon-expanded stents typically prepared from stainless steel, cobalt chrome, and others); other vascular devices (e.g., grafts, catheters, valves, artificial hearts, heart assist devices); implantable defibrillators, especially defibrillator leads; blood oxygenator devices (e.g., tubing, membranes); surgical devices (e.g., sutures, staples, anastomosis devices, vertebral disks, bone pins, suture anchors, hemostatic barriers, clamps, screws, plates, clips, vascular implants, tissue adhesives and sealants, tissue scaffolds); membranes; cell culture devices; chromatographic support materials; biosensors; shunts for hydrocephalus; wound management devices; endoscopic devices; infection control devices; orthopedic devices (e.g., for joint implants, fracture repairs); dental devices (e.g., dental implants, fracture repair devices), urological devices (e.g., penile, sphincter, urethral, bladder, prostrate, vaginal, fallopian, and renal devices, and catheters); colostomy bag attachment devices; ophthalmic devices (e.g., ocular coils); glaucoma drain shunts; synthetic prostheses (e.g., breast); intraocular lenses; respiratory, peripheral, cardiovascular, spinal, neurological, dental, gastro-intestinal, gastro-esophageal (e.g., for Barrett's Esophagus or pre-cancerous esophageal tissue or cells), ear/nose/throat (e.g., ear drainage tubes); renal devices; iliac devices; cardiac devices; aortic devices (e.g., grafts or stents); and dialysis (e.g., tubing, membranes, grafts).

Examples of useful devices include urinary catheters (e.g., surface-coated with antimicrobial agents such as vancomycin or norfloxacin), intravenous catheters (e.g., treated with additional antithrombotic agents such as heparin, hirudin, and/or coumadin), small diameter grafts, vascular grafts, artificial lung catheters, atrial septal defect closures, electro-stimulation leads for cardiac rhythm management (e.g., pacer leads), glucose sensors (long-term and short-term), degradable, non-degradable, or partially degradable coronary stents, blood pressure and stent graft catheters, birth control devices, benign prostate and prostate cancer implants, bone repair/augmentation devices, breast implants, cartilage repair devices, dental implants, implanted drug infusion tubes, intravitreal drug delivery devices, nerve regeneration conduits, oncological implants, electrostimulation leads, pain management implants, spinal/orthopedic repair devices, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, hemodialysis devices, catheter cuff, anastomotic closures, vascular access catheters, cardiac sensors, uterine bleeding patches, uterine stent or stent-like devices, cervix treatment devices, urological catheters/stents/implants, gastro-esophageal stents, treatments for lower esophageal sphincter, in vitro diagnostics, aneurysm exclusion devices, and neuropatches.

Examples of other suitable devices include, but are not limited to, vena cava filters, urinary dialators, endoscopic surgical tissue extractors, endoscopic drug or fluid delivery devices, atherectomy catheters or devices, imaging catheters or devices (e.g., Intravascular Ultrasound (IVUS), Magnetic Resonance Imaging (MRI), or Optical Coherence Tomography (OCT) catheters or devices), thrombis and/or clot extraction catheters or devices (e.g., thrombectomy devices), percutaneous transluminal angioplasty catheters or devices, PTCA catheters, stylets (vascular and non-vascular), guiding catheters, drug infusion catheters, esophageal stents, pulmonary stents, bronchial stents, circulatory support systems, angiographic catheters, transition sheaths and dilators, coronary and peripheral guidewires, hemodialysis catheters, neurovascular balloon catheters or devices, tympanostomy vent tubes, cerebro-spinal fluid shunts, defibrillator leads, percutaneous closure devices, drainage tubes, thoracic cavity suction drainage catheters, electrophysiology catheters or devices, stroke therapy catheters or devices, abscess drainage catheters, biliary drainage products, dialysis catheters, central venous access catheters, and parental feeding catheters or devices.

Examples of medical devices suitable for the present invention include, but are not limited to catheters, implantable vascular access ports, blood storage bags, vascular stents, blood tubing, arterial catheters, vascular grafts, intraaortic balloon pumps, sutures (e.g., cardiovascular), total artificial hearts and ventricular assist pumps, extracorporeal devices such as blood oxygenators, blood filters, hemodialysis units, hemoperfusion units, plasmapheresis units, hybrid artificial organs such as pancreas or liver and artificial lungs, as well as filters adapted for deployment in a blood vessel in order to trap emboli (also known as “distal protection devices” or “distal embolic protection devices”), whereas progesterone and optionally Vitamin E or conjugated linoleic acid can provide a therapeutic effect as described herein.

Numerous devices known to the art can be used to deliver the progesterone-containing composition. Such devices include, but are not limited to: Wolinsky double-style balloon (e.g., USCI Division, CR Bard, Inc. Billerica, Mass.); microporous balloon (e.g., 15 cm holes, 0.4-0.8 μm post sizes Cordis Corp, Miami Lakes, Fla.); multichannel balloon (e.g., Boston Scientific, Watertown, Mass.); Infusosleeve (e.g., Local Med); dispatch catheter (e.g., SciMed); hydrogel balloon (e.g., Boston Scientific); needle injection (e.g., BMI Inc, Oberpfaffenhofen, Germany); OROS platform (ALZA Corp/Johnson and Johnson Corp.); Macroflux platform (Macroflux Corp.); and microcatheter (e.g., Terumo Medical Corp), or derivatives, modifications, or alternative versions of these or combinations thereof.

The coated device can be composed of any suitable biocompatible, bioerodable, and/or bio-tolerant material including, but not limited to, gold, tantalum, iridium, platinum, nitinol, stainless steel, platinum, titanium, tantalum, nickel-titanium, cobalt-chromium, magnesium, ferromagnetic, nonferromagnetic, alloys thereof, fiber, cellulose, various biodegradable or non-biodegradable polymers, or combinations thereof. For example, the device can be composed of MP35N or MP20N (trade names for alloys of cobalt, nickel, chromium, and molybdenum, Standard Press Steel Co., PA). The coated device can be a metal (e.g., transition, actinide, or lanthanide metal). The coated device can be non-magnetic, magnetic, ferromagnetic, paramagnetic, or superparamagnetic. The coated device can further include strength-reinforcement materials that include but are not limited to, thickened sections of base material, modified surface properties (e.g., for promotion of endothelial progenitor cells), modified geometries, intermediate material, coating, fibers (such as composites, carbon, cellulose or glass), kevlar, and/or other material.

The coated device can be composed of a biodegradable, a bioerodable, a non-biodegradable material, a non-bioerodable material, or a combination thereof. The coated device can be permanent or temporary. A temporary device can be resident for a period of time such as about 15 days, about 30 days, about 60 days, about 90 days, or longer.

Suitable non-biodegradable polymers include: polyetheretherketone (PEEK), PEEK derivatives, polyethyleneteraphthalate, polyetherimide, polymide, polyethylene, polyvinylfluoride, polyphenylene, polytetrafluoroethylene-co-hexafluoropropylene, polymethylmethacrylate, polyetherketone, poly (ethylene-co-hexafluoropropylene), polyphenylenesulfide, polycarbonate, poly (vinylidene fluoride-co-hexafluoropropylene), poly (tetrafluoroethylene-co-ethylene), polypropylene, and polyvinylidene fluoride.

Suitable biodegradable materials include: polycaprolactone, poly (D,-lactide), polyhydroxyvalerate, polyanhydrides, polyhydroxybutyrate, polyorthoesters, polyglycolide, poly (L-lactide), copolymes of lactide and glycolide, polyphosphazenes, and polytrimethylenecarbonate. One example of a device of biodegradeable material is the Igaki-Tamai stent.

Stent

Devices which are particularly suitable include vascular stents, such as self-expanding stents and balloon expandable stents. All types of stents, including those known in the art, may be utilized in association with the present invention. Generally, a stent is a tube-like device made of metal or plastic that is inserted into a vessel or passage to keep the lumen open and prevent closure due to a stricture or external compression. The style and composition of the stent may comprise any biocompatible material, or non-biocompatible material with a biocompatible, bioerodable, and/or biodegradable coating, or coating having the ability to support a vessel. The stent can have a mesh structure and be produced from, for example, metal, plastic, and/or fibers (e.g., PTFE, polypropylene, polyethylene, PEEK, PEEK derivatives, silk, cotton and the like), or combinations of these and other materials. The stent can have microscopic or macroscopic pores in the stent surface, or in the body of the stent, that serve as reservoirs for the progesterone-containing composition. The stent can be of a variety of designs, including but not limited to, slotted, hinged, braided, etc.

Examples of self-expanding stents and/or suitable balloon-expandable stents useful in the present invention are illustrated in U.S. Pat. No. 7,186,789; U.S. Pat. No. 7,163,555; U.S. Pat. No. 4,655,771; U.S. Pat. No. 4,954,126; U.S. Pat. No. 5,061,275; U.S. Pat. No. 4,733,665; U.S. Pat. No. 4,800,882; U.S. Pat. No. 4,886,062; U.S. Patent App. Pub. No. 2007/0032856; U.S. Patent App. Pub. No. 2006/0287709; U.S. Patent App. Pub. No. 2006/0271165; U.S. Patent App. Pub. No. 2005/0070996; U.S. Patent App. Pub. No. 2004/0215315; U.S. Patent App. Pub. No. 2004/0215314; U.S. Patent App. Pub. No. 2004/0133270; and U.S. Patent App. Pub. No. 2004/0093064, the contents of each of which is hereby incorporated by reference.

The drug-eluting stents described herein are applicable to all vascular and non-vascular stent applications in the body including coronary, peripheral, carotid, and neurological arterial system. The drug-eluting stents described herein are also applicable to all vascular stent applications in the body including coronary, peripheral and neurological venous, endocrine, gastro-intestinal, gastro-esophageal, limbic, or hormonal system. Stents are commonly used, for example, to keep blood vessels open in the coronary arteries; in the esophagus for strictures or cancer; the ureter to maintain drainage from the kidneys; or the bile duct for pancreatic cancer or cholangiocarcinoma. Stents are also commonly utilized in other vascular and neural applications to keep blood vessels open and provide structural stability to the vessel. The coated stents described herein can be used to provide support to weakened, diseased, or problematic structures (e.g., heart valves, venous valves, heart wall, nasal sinuses, arteries, urinary tracts, reproductive tracts, airways, digestive tracts, ear canal). The coated stents described herein can be used as vessel grafts or vessel extensions. Stents are usually inserted under radiological guidance and can be inserted percutaneously through, for example, the femoral, brachial, or radial approach. The stent or device can also be inserted intramuscularly (e.g., injected into a muscle via an open surgical procedure such as open heart surgery, or via a minimally invasive procedure). The coated stent described herein can also be utilized in the treatment of vulnerable plaque, such as thin fibrous-capped atheromatic vulnerable lesions. Treatment of vulnerable plaque with a coated stent described herein can provide desirable drug and release kinetics with site specificity.

Stents constructed with any suitable material may be utilized with the progesterone-containing composition described herein. Stents can be made from, for example, gold, tantalum, iridium, platinum, nitinol, stainless steel, platinum, titanium, tantalum, nickel, cobalt, chromium, magnesium, ferromagnetic, nonferromagnetic, alloys thereof, fiber, cellulose, various biodegradable or non-biodegradable polymers, various bioerodadable or non-bioerodable polymers, other polymers or combinations thereof. For example, the device can be composed of MP35N or MP20N (trade names for alloys of cobalt, nickel, chromium, and molybdenum, Standard Press Steel Co., PA), Elgiloy (cobalt chromium alloy), 316L stainless steel, Biodur 108 (high nitrogen stainless steel), L-605 (cobalt chrome alloy), Elastinite (Nitinol), nickel-titanium alloy, or platinum-iridium alloy.

One example of a stent that may be utilized with the present invention includes weaved materials or braided materials such as metals (e.g. nitinol), plastics (e.g. polypropylene, polyethylene, PTFE, ePTFE, polyester, PEEK) and fibers (e.g. cotton, silk, kevlar), or combinations thereof. A mesh covering can be included over or within the stent, where the mesh is composed of the same or different materials as the balance of the stent. Examples of various polymers used in forming a mesh covering or insert include, for example, poly(methyl(meth)acrylate (“PMMA”), ethylenevinylalcohol (“EVAL”), poly(butyl(meth)acrylate) (“PBMA”), biodegradable polymers (i.e., Poly(glycolic acid) (“PGA”) and poly(L-lactic acid) (“PLLA”), polyethylene glycol (“PEG”), hyaluronic acid (“HA”), polyester amide (“PEA”), poly(glycerol-sebacate) (“PGS”) (developed by Yadong Wang, MIT), nanoscale structures of carbon, acetal copolymer, acetal homopolymer, acrylonitrile butadiene styrene, ABS and polycarbonate, nylon, polyamide, polyacrylate, polyaryl sulfone, polycarbonate, polyetherketone, PEEK, PEEK derivatives, polyetherimide, polyether sulfone, polyethylene terephthalate, polyimide, polyphenylene oxide, polyphenylene sulfide, polypropylene, polysulfone, polyurethane, polyvinyl chloride, styrene acrylonitrile and other suitable polymers. It is contemplated that the progesterone-containing composition can be coated on at least a portion of the stent; and/or on, in, and/or underneath the mesh covering or insert; or a combination thereof.

One example of a suitable stent is the Sorin Carbostent, which is 316 LVM stainless steel permanently coated with a thin film of turbostatic carbon. Other examples of suitable stents include Multi-Link Penta™, Multi-Link Tetra™, Multi-Link Vision™, Multi-Link Frontier™ (Advanced Cardiovascular Systems); BX Velocity™ (Cordis Corp., Fla.); and Express Stent (Boston Scientific Inc., MA).

One embodiment of the present invention includes single strand stents. Single strand stents generally include a single strand of a suitable material (e.g., gold, nitinol, stainless steel, biodegradable polymers, plastic and/or combinations thereof) that is shaped to provide a structural scaffolding, which supports the walls of the host tissue surrounding it. In various embodiments, at least a portion of the single strand stents are coated with a progesterone-containing composition described herein. The single strand stent can include metallic or polymeric spring, ring or any wire shape support that collapses for insertion into a catheter and then expands when deployed from the catheter to hold the stent against the blood vessel wall. The spring, ring or wire can be made out of any suitable material, such as gold, nitinol, stainless steel, polymeric material, rubber, etc. The material in these various embodiments for any or all of the components can also be biodegradable, bioresorbable, or bioerodable, either in total or in part. The spring, ring or wire is generally made so that it can collapse on its side and elongate to reduce its size so as to fit within a delivery catheter.

Coating

The device coating can be composed of one layer or multiple layers. One layer will consist of a drug-eluting coating that contains progesterone and, optionally, additional therapeutic agents, such as vitamin E and/or conjugated linoleic acid. In various embodiments, there are more than one drug-eluting layers, each containing progesterone and/or additional therapeutic agents. The device can also be coated with other layers, such as a primer layer, barrier layer, and/or topcoat layer. The primer layer, also known as an adhesion layer, generally prepares the exposed stent surface for the drug-eluting coating. The barrier layer and cap layer can provide an additional layer(s) of protection for the device and/or further control the elution profile of the drug(s). The barrier layer may have more, less, or substantially the same of a progesterone containing composition than the other layers. It is contemplated that one or more barrier layers can be formed between multiple drug-eluting layers. For example, a first barrier layer can be positioned between a first and a second drug-eluting layer; or a first and second barrier layer can be positioned between a first, a second, and a third drug-eluting layer, respectively.

Preferably the coating(s) is biodegradable and/or bioerodable. A biodegradable, bioerodable, and/or other coating can be combined with a slow release agent that allows the progesterone or progesterone composition to act for an extended time period.

Preferably, the progesterone-containing composition is a component of the drug-eluting layer(s) of the device. But it is also contemplated that the progesterone-containing composition can be a component of other layers, such as an adhesion layer, a barrier layer, and/or a cap layer.

The coated device described herein can contain more than one coating layer. In one such embodiment, for example, the coating comprises at least two different layers. For example, a primer layer is applied; after which one or more drug-polymer layers are coated, each with or without progesterone, and each with or without additional therapeutic agents; after which a barrier topcoat layer is applied. These different layers, in turn, can cooperate in the resultant composite coating to provide an overall release profile having certain desired characteristics. In some embodiments, the composition is coated onto the device surface in one or more applications of a single composition that contains progesterone, together with optional additional therapeutic agent(s). A pretreatment layer or layers can be first applied to the surface of the device, wherein subsequent coating with the composition may be performed onto the pretreatment layer(s).

A primer layer, or adhesion layer, can be disposed between other layers, such as a barrier layer or drug-eluting layer, and the material of the device. The adhesion layer can enhance the adhesion between a surface of a device (e.g., a metallic surface of a stent) and a progesterone-containing composition. Examples of adhesion coatings/additives include a polyurethane, a phenoxy, poly(lactide-co-glycolide), polylactide, polysulfone, polycaprolactone, an adhesion promoter, silane coupling agents, photografted polymers, epoxy primers, polycarboxylate resins, Parylene™ coatings, hyaluronan coatings, plasma treatments, argon treatments, physical roughening of the surface, physical modifications of the surface, nanomolecular treatments, or combinations thereof. It is further noted that the pretreatment compositions may be used in combination with each other or may be applied in separate layers to form a pretreatment coating on the surface of the medical device. The adhesion layer can be applied by any suitable coating method such as spraying, dipping, painting, ionizing, atomizing, brushing or dispensing. The adhesion layer can be dried at room temperature or at an elevated temperature suitable for driving off any solvents. A nitrogen, dehumidifying, and/or vacuum environment can be used to assist the drying process.

The progesterone-containing composition can be applied directly to the surface or interior of a device, or alternatively, to the surface or interior of a surface-modified device, by dipping, spraying, brushing, ultrasonic deposition, compressed fluid, supercritical fluid processing, supercritical carbon dioxide, or using any other conventional or non-conventional technique. The suitability of the progesterone-containing composition for use on a particular material, and in turn, the suitability of the coated composition can be evaluated by those skilled in the art, given the present description.

The progesterone-containing composition is usually applied in conjunction with a polymer and suitable solvent (e.g., ethanol, chloroform, or tetrahydrofuran (THF)). The drug-polymer solution can be dried by evaporating the solvent after application. The drying can be performed at room temperature or an elevated temperature. The drying can be performed at standard pressure or under vacuum. A nitrogen environment or other controlled environment can also be used. For example, the drug-polymer solution can be dried by driving off solvents in the solution via heating at an elevated temperature in an inert ambient nitrogen environment under vacuum. Alternatively, the drug-polymer solution can be dried by evaporating the majority of the solvent at room temperature, and further drying the solution in a vacuum environment between a temperature of about 25° C. to about 45° C. or higher to extract any pockets of solvent buried within the drug-polymer coating. Additional coats can be added to thicken the drug coating and/or to increase the drug dosage. Additional layers can be applied over the dried drug polymer; examples of such additional layers including a barrier layer, a cap layer, another drug-polymer layer, or combinations thereof. The polymer layer, as well as other layers, can be applied to at least a portion of the interior surface and/or the exterior surface of the stent framework.

Compressed fluids and supercritical fluids, and those involving compressed carbon dioxide can be used for polymer synthesis and processing (see e.g., Example 11). Compressed fluids and supercritical fluids can be used as a transfer agent to introduce progesterone or a progestrone-containing composition into or onto a polymer or coated onto the device. Supercritical fluid processing can be according to, for example, expansion of a supercritical solution (RESS). An RESS method of supercritical fluid processing can increase cumulative specific surface area by more than 40% (see Ind Eng Chem Res (1996) 35, 4718-4726) over conventional manufacturing techniques. Supercritical fluid processing can be according to, for example, supercritical antisolvent precipitation (SAS), gas saturated solutions (PGSS), or Gas Antisolvent process (GAS). Supercritical fluid processing can occur, for example, between about 100 to about 240 bar and about 313 to about 333 Kelvin. Supercritical fluid processing can occur, for example, between about 92 and about 240 bar and about 308 and about 333 Kelvin Carbon dioxide assisted impregnation of the progesterone or a progesterone containing composition can be used without harmful organic solvents, mechanical stresses, or elevated temperatures.

Preferably, progesterone or a progesterone-containing composition is eluted from a polymer coating covering at least a portion of the device. The polymer can provide controlled time and dosage delivery after deployment of the coated stent within a subject. Elution rates of progesterone and/or other therapeutic agents into the subject and the tissue bed surrounding the stent framework are based, at least in part, on the constituency and thickness of drug-polymer coating, the nature and concentration of the therapeutic agents, the thickness and composition of an optional capping coat, physiological factors of the anatomical location (e.g. low vs. high flow), and other factors.

The polymer coating can be made from any suitable biocompatible polymer, examples of which include ethylene vinyl alcohol copolymer (commonly known by the generic name EVOH or by the trade name EVAL); poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., PEO/PLA); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyinides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; and carboxymethyl cellulose. The coating can also be, for example, silicon foam, neoprene, santoprene, open cell foam, closed cell foam, or a combination thereof. The coating can also be, for example, any combination of the above materials and/or in combination with other biodegradable, non-biodegradable, bioerodable, non-bioerodable, biocompatible, or biocompatible material(s). The above materials can also be used as a base filler, excipient, or barrier (temporary or permanent) material in addition to, or instead of, being used as a coating.

To avoid too-rapid release of therapeutic agents from a drug-eluting device and/or to provide protection for the device, the device can include a barrier layer, or a cap layer. Generally, a barrier layer and a cap layer are similar, both providing enhanced protection and increased control of elution, where the cap layer usually refers to the outermost coating layer of the device and the barrier layer refers to intermediate layers. Where a coated device contains both a barrier layer(s) and a cap layer, the barrier layer(s) and the cap layer can be of the same material or different materials. The balance of discussion will refer to the barrier layer, but one of skill in the art will understand that such a layer may be termed a cap layer when positioned as the outermost layer of the device.

The barrier layer can be disposed on top, within, peripheral to, or below a drug-eluting layer. The barrier layer can provide, for example, additional protection from shear forces generated during device deployment. The barrier layer can aid in the control of the elution rate of progesterone and/or one or more additional therapeutic agents dispersed within or encased by the coatings. The barrier coating can be any suitable polymeric material discussed above, or known in the art, and is preferably a silicone-urethane copolymer, a polyurethane, a phenoxy, epoxy, ethylene vinyl acetate, polycaprolactone, polyimide, poly(lactide-co-glycolide), parylene, polylactide, pellathane, polysulfone, elastin, fibrin, collagen, chondroitin sulfate, a biocompatible polymer, a biostable polymer, a biodegradable polymer, a bioerodable polymer, or a combination of these or another appropriate material. For example, the barrier layer can be of parylene or its derivatives, PTFE, etc. Parylene is a highly pure, biocompatible, chemically inert coating material. The US FDA has approved the use of parylene in human implants. Parylene coatings can enhance biocompatibility and surface smoothness of medical devices. The barrier layer can also contain additional bioactive therapeutic agents. For example, to improve haemo-compatibility, anti-platelet agents (e.g., Cilostazol, Plavix, Ticlid, Ticagrelor, or derivatives thereof, etc.) can be added to the barrier coating.

The method of applying the coating composition to the device is typically governed by the geometry of the device and other process considerations. The coating(s) can be applied, for example, using any suitable application technique such as dipping, spraying, brushing, ultrasonic deposition, compressed fluid, supercritical fluid processing, supercritical carbon dioxide, or painting. A coating composition can be provided in any suitable form, e.g., in the form of a true solution, or fluid or paste-like emulsion, mixture, dispersion or blend. The coated composition will generally result from the removal of solvents or other volatile components and/or other physical-chemical actions (e.g., heating or illuminating) affecting the coated composition in situ upon the surface. The coating material can be dissolved or suspended in a suitable solvent such as isopropyl alcohol, ethanol, or methanol, before application, applied, and then dried. The coating can be subsequently cured by, for example, evaporation of the carrier solvent. The coating material may be dried, for example, in air, at room or elevated temperature, and optionally with the assistance of vacuum and/or controlled humidity. In some cases, ultraviolet radiation (UV), gamma radiation or e-beam irradiation may be used to aid in curing or cross-linking the coating material.

The progesterone-containing composition can be coated on a device through, for example, an evaporation process or some other known method. The solvent evaporation process entails combining polymeric materials, the therapeutic agent(s) (i.e., progesterone and/or additional therapeutic agents), and a solvent (e.g., tetrahydrofuran) forming a mixture. The mixture can then be applied to the device by, for example, spraying the solution onto the device, injecting into reservoirs in the device, or dipping the device into the solution. After the mixture has been applied, the device can be subjected to a drying process, during which, the solvent evaporates and the polymeric material and therapeutic agent form a thin film on the device. In various embodiments, therapeutic agent(s) in addition to progesterone can be added to the layer(s).

It is understood that one or more additional layers may be applied to the coating layer(s) that include progesterone. Such layer(s) can be utilized to provide a number of benefits, such as biocompatibility enhancement, delamination protection, durability enhancement, improved pharmacokinetics, improved pharmocodynamics, improved tissue concentration, improved deliverability, improved absorption, improved adsorption, and/or therapeutic agent(s) release control, to just mention a few. In another embodiment, one or more of the pretreatment materials may be applied as a topcoat or cap layer. Additionally, biocompatible topcoats (e.g. heparin, collagen, phosphorylcholine, extracellular matrices, cell receptors, hydroxyapatite, etc.) can be applied to the coating composition of the present invention. Such biocompatible topcoats may be adjoined to the coating composition of the present invention by utilizing photochemical or thermochemical techniques known in the art. Additionally, release layers may be applied to the coating composition of the present invention as a friction barrier layer or a layer to protect against delamination. Examples of biocompatible topcoats that may be used include those disclosed in U.S. Pat. Nos. 4,979,959 and 5,744,515.

Optionally, a hydrophilic topcoat can be provided. Such topcoats may provide several advantages, including providing a relatively more lubricious surface to aid in medical device placement in situ, as well as to further increase biocompatibility in some applications. Examples of hydrophilic agents that may be suitable for a topcoat in accordance with the invention include polyacrylamide(36%)co-methacrylic acid(MA)-(10%)co-methoxy PEG1000MA-(4%)co-BBA-APMA compounds such as those described in example 4 of U.S. Patent App. Pub. No. 2002/0041899; photoheparin such as described in example 4 of U.S. Pat. No. 5,563,056; and a photoderivatized coating as described in Example 1 of U.S. Pat. No. 6,706,408, the contents of each of which is hereby incorporated by reference.

Optionally, the progesterone coating can be used in combination with another coating, such as a radiopaque coating, fluoroscopic imaging coating, and liposomal delivery coating. If combined for radiopacity, the progesterone-containing composition can be compounded with material such as tantalum, barium sulfate, bismuth oxychloride, bismuth subcarbonate, tungsten, gold bismuth trioxide, or other appropriately dense radiopaque material.

In some embodiments, the topcoat may be used to control or effect the elution rate of progesterone and/or one or more other therapeutic agents from a medical device surface. For example, topcoats may be described as the weight of the topcoat relative to the weight of the underlying therapeutic agent(s) containing layer. For example, the topcoat may be about 1 percent to about 50 percent by weight relative to the underlying layer. In some embodiments, the topcoat may be about 2 percent to about 25 percent by weight relative to the underlying layer. Optionally, in some embodiments, the topcoat may be about 5 percent to about 12 percent by weight relative to the underlying layer. It will be understood by one skilled in the art that such percentages are exemplary and do not serve to limit the invention.

Further, in some embodiments, progesterone and/or one or more other therapeutic agents may be provided in a topcoat (sometimes referred to as a topcoat therapeutic agent(s)). The topcoat therapeutic agent(s) may be the same as or distinguishable from the therapeutic agent(s) included in an underlying layer. Providing therapeutic agent(s) within the topcoat allows for the therapeutic agent(s) to be in contact with surrounding tissue in situ while providing a longer release profile compared to coating compositions provided without topcoats. Such topcoats may also be used to further control the elution rate of a therapeutic agent(s) from a medical device surface, such as by varying the amount of therapeutic agent(s) in the topcoat. The degree to which the therapeutic agent(s) containing topcoat affects elution will depend on the specific therapeutic agent(s) within the topcoat as well as the concentration of the therapeutic agent(s) within the topcoat. One example of a topcoat material is parylene and/or its derivatives (e.g., PTFE, ePTFE). Parylene is biocompatible, chemically inert coating material approved for use on human implants. Parylene coatings can enhance biocompatibility and surface smoothness of medical instruments.

Any suitable amount of a therapeutic agent may be included in the topcoat. For example, the upper limit of the amount of agent in the topcoat may be limited only by the ability of the topcoat to hold additional agent. In some embodiments, the agent may comprise about 1 to about 75 percent of the topcoat. Optionally, the agent may comprise about 5 to about 50 percent of the topcoat. In yet other embodiments, the agent may comprise about 10 to about 40 percent of the topcoat.

A further example of a coating composition embodiment may include a configuration of progesterone and/or one or more other therapeutic agents within an inner matrix structure, for example, within or delivered from a degradable encapsulating matrix or a microparticle structure formed of semipermeable cells and/or degradable polymers. One or more inner matrices may be placed in one or more locations within the coating composition and at one or more locations in relation to the substrate.

The overall weight of the coating upon the surface may vary depending on the application. However, in some embodiments, the weight of the coating attributable to the therapeutic agent(s) is in the range of about 1 μg to about 10 mg of therapeutic agent(s) per cm² of the effective surface area of the device. “Effective” surface area is understood as the surface amenable to being coated with the composition itself. For a flat, nonporous, surface, for example, this will generally be the macroscopic surface area itself, while for considerably more porous or convoluted (e.g., corrugated, pleated, or fibrous) surfaces, the effective surface area can be significantly greater than the corresponding macroscopic surface area. In various embodiments, the weight of the coating attributable to the therapeutic agent(s) is between about 0.005 mg and about 10 mg, and in some embodiments between about 0.01 mg and about 1 mg of therapeutic agent(s) per cm² of the gross surface area of the device. This quantity of therapeutic agent(s) is generally required to provide desired activity under physiological conditions. For example, a drug eluting stent may have a gross surface area between 10 μm and 1000 μm, or between 250 μm and 750 μm. In some embodiments, a drug eluting stent or medical device will have enough drug to elute over a 30, 45 or 60 day period of time, for example, enough time for endothelialization to occur.

In turn, in various embodiments, the final coating thickness of a coated composition will typically be in the range of about 0.1 μm to about 100 μm, and in some embodiments, between about 0.5 μm and about 25 μm. This level of coating thickness is generally required to provide an adequate concentration of drug to provide adequate activity under physiological conditions.

Suitable additives to the polymer coating include cross-linking agents, dispersants (wetting agents) and plasticizers. Cross linking agents (e.g., acylamine, amidoformate) can provide structural integrity to the coating. Dispersants (i.e., wetting agents) can enhance dispersion of the polymer, to make the distribution of components of the solution more uniform, and ionic or non-ionic surfactants are suitable. A plasticizer can improve the mechanical characteristics of the coating. Plasticizers including linear polymers such as polyaether may be used.

The coating can substantially cover the entire device surface or only a portion of the device. For example, a stent coating can be on the outside section, inner lumen, struts only, sides of struts, mesh, links, rings, wires, crowns, hoops, embedded within pockets within the struts or structure, on the distal, middle, and/or proximal edge of the device, and in various patterns such as a helix, double helix, triple helix, multi-helix, striated pattern, spiral pattern, curved pattern, patches, polka dotted pattern, or any other geometric and/or random pattern and/or any combination of these or other configurations

Where the progesterone-containing composition (and optional additional therapeutic agent(s)) is coated on the outside, or within, of an implanted device, the composition can be delivered directly into the tissue contacting the device surface (e.g., vessel wall) or via osmosis within the fluid environment. Inclusion of vitamin E in the device coating can facilitate delivery of progesterone and/or additional therapeutic agents, such as conjugated linoleic acid, into the vessel wall and/or can improve its therapeutic properties due to its biochemical capabilities, as discussed herein. Where the progesterone-containing composition is coated on the inner surface of, or within, an implanted device, such that for example blood flows through it, the progesterone-containing composition can be delivered directly into the blood stream.

The progesterone-containing coating can dissolve quickly or slowly over time. The coating can be designed to dissolve naturally in the body, or be activated by, for example, UV light, visible light, non-visible light, ultrasound, infrared, light, heat, ph change, radio frequency signal, magnetic signal, a chemical or agent, any combination of any of these, or some other form of activation.

Those skilled in the art will appreciate the manner in which the combined effect of these various layers can be used and optimized to achieve various effects in vivo.

In Need Thereof

The subject to which the progesterone-containing composition, coated device, or delivery device is administered can be any subject in need of a therapeutic treatment. Therapeutic treatment is understood to also include prophylactic treatment. Preferably, the subject is a mammal, reptile, or avian. More preferably, the patient is a human. Furthermore, the composition delivery system or coated device can be implanted in any location to which it is desired to effect a local therapeutic response. A subject in need thereof includes, but is not limited to, a subject diagnosed with, at risk for, or at risk for reoccurrence of conditions including coronary restenosis, cardiovascular restenosis, angiographic restenosis, arteriosclerosis, neointimal hyperplasia, neoplastic progression, dysplastic, non-dysplastic or partially dysplastic Barrett's esophagus, vulnerable plaque, thrombosis, and/or related diseases and conditions. A determination of the need for treatment will typically be assessed by a history and physical exam consistent with the disease or condition.

Applications

The progesterone-containing composition and/or coated device can be used for a variety of applications, including but not limited to, coronary, cardiac, peripheral, carotid, gastro-intestinal, gastro-esophageal, prostate, uterine, and/or neurovascular applications. For example, the progesterone-containing composition and/or coated device can be used for thromboresistance, haemocompatibility, and biocompatibility in vascular grafts and heart valves.

The progesterone-containing composition and/or coated device are effective to achieve a variety of effects in a variety of applications. The progesterone-containing composition, coated device, and/or delivery device can provide for one or more of the following: repel, slow, or eliminate neo-intimal hyperplasia, or new cell growth; prevent, slow, or eliminate the growth or regrowth of fatty tissue and cholesterol deposits; prevent, slow, or eliminate new lesion growth or lesion regrowth, such as in restenosis; prevent, slow, or eliminate tumor growth and/or tumor-like growths, such as a lesion in an blood vessel; minimize or prevent thrombus formation and reduction of inflammatory responses at, near, or downstream from the site of composition delivery or device implantation; normalize blood clotting and vascular tone; mediate an anti-proliferative signal cascade; support a healthier type of neointimal formation (e.g. endothelial cell growth and/or lining of the arterial lumen); promote collagen development; attract increased levels of collagen in the proteoglycan matrix; decrease platelet adherence on a surface of an implanted device with less neutrophils and monocysts, resulting in less thrombus and/or leukocyte adherence; promote smooth endothelial lining; promote thinner neointimal layer; contribute to inhibition of smooth muscle cell proliferation and/or neointimal growth; act as an antiinflammatory agent and regulator of the immune response; reduce, eliminate, prevent, or minimize a harmful effect of vulnerable plaque; repel cholesterol, fatty deposits, calcium, fatty esters and/or other constituents of potential lesion foundations; eradicate or minimize dysplastic Barrett's esophagus; and/or decrease the rate of neoplastic progression.

Restenosis is a condition related to cell proliferation. Where a device, such as a guide wire, catheter, balloon, and/or stent, is used to access and open a blood vessel passageway, it can injure endothelial cells lining blood vessel and the smooth muscle cells surrounding the tissue. An injured site is vulnerable until the endothelium is mature. Within the first 24 hours of injury, smooth muscle cells, leukocytes, and red blood cells are present, after which there is mostly smooth muscle cells. Endothelium begins to form within one week. After four to five weeks, there exists more mature endothelium, which can function to, for example, keep the arteries clear and lubricious. But wound repair mechanisms result in exposed smooth muscle cell proliferation and migration, again narrowing the opening in the vessel in, for example, three to six months after angioplasty. The anti-proliferation effects of progesterone in the compositions described herein can function to counter such restenosis-related excess cell growth.

The progesterone-containing composition and/or coated device can prevent proliferation and migration of certain repair entities, such as white blood cells and/or cytokines, to the site of injury, thereby preventing thrombus-like reactions, neointimal hyperplasia, and/or restenosis.

The progesterone-containing composition and/or coated device can treat bifurcated lesions and/or ostial lesions (e.g., renal ostial, aortic ostial and/or iliac ostial locations).

The progesterone-containing composition and/or coated device can repel cholesterol, fatty deposits, calcium, fatty esters and/or other constituents of potential lesion foundations. As known in the art, lesions may start as a fatty streak, building over time. By providing a surface of a material with a composition that repels cholesterol, fatty deposits, calcium, fatty esters, and/or other constituents of potential lesion foundations, then this surface can remain lesion free, or at least not grow beyond a reasonable size, such that it occludes the artery, vein or area of interest being treated.

The progesterone-containing composition and/or coated device can block potentially dangerous effects of estrogen. Estrogen in the uterus causes proliferation of the cells. Under the influence of estrogen, uterine cells multiply faster; but progesterone produced with ovulation serves to inhibit the increased cell multiplication. Progesterone is understood to cause the cells to mature and enter into a secretory phase that causes the maturing of the uterine lining. Such anti-proliferative effects are useful for treatment of the conditions described herein.

The progesterone-containing composition and/or coated device can prevent and/or remove cholesterol deposits or build up. One of the chief causes of coronary heart disease is not cholesterol per se, but oxidized cholesterol. As such, increases in cholesterol oxidation increases the risk of coronary heart disease. The progesterone-containing composition, along with optional agents such as vitamin E and/or conjugated linoleic acid, can serve to decrease cholesterol oxidation.

The progesterone-containing composition can be used in conjunction with biosynthetic blood vessels. Some such small diameter biosynthetic blood vessels are developed from collagen tubes and may become colonized with vascular cells in situ. The progesterone-containing composition can be infused within the collagen tubes, coated on the outside and/or inside, compounded in multiple layers, and/or compounded with other chemicals shown to be effective at preventing or reducing colonization of unwanted vascular and/or non-vascular cells in biosynthetic blood vessels in situ. The collagen framework of the biosynthetic blood vessels can be embedded with an amount of the progesterone-containing composition effective to allow some vascular endothelium growth but prevent over-proliferation and/or uncontrolled growth. Saphenous vein grafts are another example of vessels which can benefit from such a treatment, whether they are biosynthetic, synthetic, animal, human, or a combination thereof. The progesterone-containing compound can be employed within the lumen, outside of the lumen, into the lumen walls (i.e. between the lumen layers), and/or in any combination thereof

The progesterone-containing composition can be used to reduce or eliminate Cardiac allograft vasculopathy (CAV). CAV is a long-term complication of heart transplantation manifested by a unique and unusually accelerated form of coronary disease affecting both intramural and epicardial coronary arteries and veins (see Weis and Shceidt (1997) Circulation 96(6), 2069-77). Methods using progesterone or a progesterone-containing composition may have helpful and beneficial effects, such as reduced rates of restenosis and/or thrombosis and/or reduction or prevention of allograft rejection and vasculopathy in cardiac transplant recipients.

It is understood that various progesterone-containing compositions and coated devices described herein could be utilized in a variety of targeted therapeutics, tissue and cellular imaging, tissue engineering, and biosensors and diagnostics applications.

Device Delivery

In use, the coated device (e.g., a drug eluting stent) or delivery device can be deployed using conventional techniques. Once in position, the therapeutic progesterone-containing composition gradually diffuses into adjacent tissue at a rate dictated by the parameters associated with, for example, the polymer coat layer. The total dosage that is delivered is of course limited by the total amount of the therapeutic active agent(s) that had been loaded within the coating. The therapeutic active agent(s) is selected to treat the deployment site and/or locations downstream and/or immediate adjacent thereof. For example, deployment in one or more of the coronary arteries can serve to deliver the therapeutic composition to the arterial area of the implant, but can also be used to allow some or all of the composition to travel to and treat the surrounding area or the distal component (i.e., downstream) of the vessel. If injected, pressed, embedded, and/or pressurized into the wall of the artery via a drug infusion device, balloon, or other technique, the composition can be used to treat via access of the advential layer of the arteries and/or the internal lumen of the artery and/or external to the artery via the heart muscle tissue (myocardium). As another example, deployment in the carotid artery can serve to deliver the therapeutic composition to the arterial area of the implant, but can also be used to allow some or all of the composition to travel to and treat the surrounding area of the implant, the area distal to the implant, the neurovasculature, or brain.

In a typical procedure to implant a stent, a guide wire is advanced through the subject's vascular system by well known methods so that the distal end of the guide wire is advanced through and/or past the plaque or diseased area. Prior to implanting the stent, the cardiologist may wish to perform an angioplasty procedure or other procedure (e.g., atherectomy) to open the lesioned vessel region and remodel the diseased area, or view via intravascular ultrasound (IVUS). Thereafter, the stent delivery catheter assembly is advanced over the guide wire so that the stent is positioned in the target area. The stent position may be monitored, for example, using radiopaque markers and/or radiopaque fluid with associated x-ray imaging systems. Once in place, the expandable member or balloon is inflated by well known means so that it expands radially outward and in turn expands the stent radially outward until the stent is apposed to the vessel wall. The expandable member is then deflated and the catheter withdrawn from the subject's vascular system. The guide wire typically is left in the lumen for post-dilatation procedures, if any, and subsequently is withdrawn from the subject's vascular system. The stent serves to hold open the artery after the catheter is withdrawn. Due to the formation of a typical stent from an elongated tubular member, the transverse cross-section is typically relatively flat, so that when the stent is expanded, it is pressed into the wall of the artery and as a result causes only minimal to no interference with the blood flow through the artery. The stent is pressed into the wall of the artery and eventually can be covered with endothelial cell growth which further minimizes blood flow interference.

The progesterone-containing composition or device described herein can be delivered intravascularly (e.g. within and into the coronary arteries). In conjunction with intravascular delivery, or in isolation from intravascular delivery, the progesterone-containing composition or device described herein can be delivered into the space between the perivascular tissue and artery, adjacent to the perivascular tissue, or into the adjacent muscle capsule. For example, a progesterone-containing composition can be included in or on a wrap designed for an artery, organ, vessel or lumen, such as a confluent endothelial cell seeded matrix (e.g., Vascugel, Pervasis Therapeutics).

Various other delivery systems are known in the art and can be used to administer the agents of the invention. Moreover, these and other delivery systems may be combined and/or modified to optimize the administration of the agents of the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Controlled Release Layer Coating

For primer base coating of a stent, 0.5 g copolymer of ethylene and vinyl alcohol is put into 10 ml N,N-dimethylacetamide. The mixture is dispersed at 80° C. and then sprayed onto stents. Thereafter the stents are dried in a vacuum oven for 2 hours at 120° C.

For barrier layer coating, Parylene is prepared by vacuum vapor deposition of 1,4-dimethylbenzene. First, 1-4-dimethylbenzene is heated to 950° C. to form dimethylbenzene dimer which cracks into monomer vapor at 680° C. later. Steel stents are then put in a deposition chamber at room temperature. Monomer vapor is introduced in the deposition chamber to form compact polymer coatings on the surface of stents. The molecular weight of polymer is estimated at 500,000.

For addition of antiplatelet-aggregation components, while the monomer steam is introduced into the substrate deposition chamber, the platelet antagonist grains (such as Cilostazol, Ticlid, Plavix and so on) are introduced into the deposition chamber. As a result, an even, compact, controllable release layer with antiplatelet aggregation function can be formed on the surface of the substrate.

### Example 2

### Coating Composition

One part composition and about 2 to about 1000 parts solvent are put into a container and dispersed. Stents are coated uniformly with the dispersed solution and then cured in a vacuum oven for 0.5-72 hours at 20-200° C. This process can be repeated with the same drug, or a different drug, dispersed in solution. Thereafter the stents are coated with 1,4-dimethylbenzene through vacuum vapor deposition. The solvents utilized are able to disperse polymers, active components, and additives uniformly. The solvents should be stable, non-reactive with the polymers, active components, and additives. The solvents should not affect on the therapeutic effect of active components; and the solvents should be volatile and readily evaporate from the coating while the coating is curing. These solvents include water; alcohol and ketone such as glycerin, isopropanol acetone, cyclohexanone butanone, ester such as ethyl acetate, butyl acetate, alkane such as n-hexane chloroform dichloromethane aromatic hydrocarbon such as benzene, methylbenzene; heterocyclic aromatic hydrocarbon such as tetrahydrofuran; and amide such as N,N-dimethylformamide and N,N-dimethylacetamide.

The polymers, active components, and additives are dispersed by stirring or ultrasonic emulsification. Thereafter, the coating is applied to the stent by dipping, spray coating, or a combination of both. The coating is cured by heat or radiation.

### Example 3

### Preparation of a Multi-Layer Progesterone-Containing Coating on a Stent by a Dip-Coating Method

A coating solution is prepared by combining and agitating a polyurethane polymer (3% wt.), progesterone (0 to 20% wt.), and THF, until thoroughly mixed. Prior to applying the layer, the stent surface is prepared and cleaned by washing it with methanol and drying it in a vacuum drier for approximately 30 minutes.

For dipping, the dry and clean stent is fully immersed into the coating solution and dried at room temperature for approximately about 5 hours in a beaker saturated with THF. This dipping/drying process is repeated about 5 times. After the fifth repetition, the stent is dried at room temperature for about 1 hour in a vacuum drier.

For spraying, the coating solution is sprayed on the cleaned stent for approximately 10 minutes and dried at room temperature. The spraying/drying process is repeated 10 times, after which the stent is dried in a vacuum drier for approximately 1 hour.

An optional second layer coating solution is prepared by mixing an optional additional therapeutic agent(s) (0 to 20% wt.) with or without progesterone in a suitable solvent (e.g., cyclohexane). The stent is then dipped into the second solution and dried at room temperature for about 1 hour, and is then dried in a vacuum drier at room temperature for about 6 hours.

### Example 4

### Whole Blood Test of Coated Stent

Three stainless steel stents, A, B, and C, are provided for the whole blood test. Stent A is left bare and had no coating applied. Stent B has a single layer coating of polyurethane, with progesterone loaded therein, applied to the stent surface. Finally, stent C has a single layer coating of polyurethane, with progesterone and vitamin E loaded therein, applied to the stent surface. All three stents are dipped in fresh rabbit blood for a period of approximately 3 minutes. After removal, the stents are examined to determine the level of thrombus formation on the stent surfaces. It is expected that stent A will be observed to have a relatively high level of thrombus formation and blood coagulation on its surface. It is also expected that stent B will be observed to have a decreased amount of thrombus formation and blood coagulation, when compared to the first stent. It is also expected that stent C will exhibit reduced amount of thrombus formation when compared to the second stent.

### Example 5

### Platelet Adhesion Test of Coated Stent

Fresh rabbit blood is mixed with 3.8 wt % sodium citrate solution at a 9:1 ratio concentration. The blood is then placed in a centrifuge and spun at 2,000 rpm for 10 minutes at 5° C. to isolate the platelets in a plasma. The plasma platelet concentration is manipulated by adding platelet-poor plasma, spun at 4,000 RPM, until a concentration level of 3×10⁵ per μl is obtained. Three stainless steel stents are then prepared as described above. The stents are incubated in the prepared plasma at 37° C. for approximately 1 hour. After removal, the stents are washed three times with a PBS solution. The stents then undergo a platelet fixation process which consists of incubating the stents in 2.5% glutaraldehyde for 4 hours. Upon completion of platelet fixation, the stents are washed in 50%, 80%, and 100% ethanol aqueous solutions. After the second washing, the samples are freeze dried for 6 hours. The stents are then examined under a scanning electron microscope to determine the platelet concentration present on each of the stent's surface. The bare stent is expected to show a uniform distribution of platelet formation on its surface. The second stent, with a progesterone-containing layer, and the third stent, with a progesterone- and vitamin E-containing coating, is expected to show a further decrease in the level of platelet adhesions.

### Example 6

### Evaluation of Inflammation of Coated Stent in Rat

A number of stainless steel strips of four varying types were prepared with different compositions of surface coating. Strip types A, B, C, and D have no coating, a progesterone-containing coating, a vitamin E-containing coating, or a progesterone and vitamin E containing coating. Strip A contains no coating. Strip B is coated with a polyurethane layer loaded with progesterone (20 wt %). Strip C is coated with a polyurethane layer loaded with progesterone (20% wt.) and vitamin E (20% wt.). Strip D is coated with a polyurethane layer loaded with vitamin E (20% wt.). The strips are prepared for implantation into male Sprague-Dawley rats.

The rats, weighing between 200-300 g, are chosen at random. The rats are first anesthetized with diethyl ether gas and secured to an operating table. One of the five types of steel strips is inserted into the back of each rat through an incision made by a scalpel. The strips are then recovered after either 14 or 30 days. The strips are recovered by anesthetizing the rats again with diethyl ether and then surgically removing a region right below where the inserted strip as well as the regions of tissue where it appears that restenosis has occurred. After removal, the strip and tissue are washed with a PBS buffer solution. The tissue is then fixed with a 4% formaldehyde solution. Each strip is then visually examined to determine the level of restenosis, if any, that had developed relative to the other strips.

It is expected that strip A, the bare strip, will show severe restenosis after 14 days. It is also expected that strip B will have reduced restenosis as compared to strip A, and that strip C will have further reduced restenosis.

### Example 7

### Elution Profile

The amount of progesterone eluted from a single layer polyurethane coating on a stainless steel sample is determined. Samples are incubated in a buffer (phosphate-buffered saline) solution at 37° C. The eluted progesterone is measured for up to about 700 hours. Intervals of measurement include 4, 8, 12, 24, 36, 48, 60, 144, 216 hours. An aliquot of the elution solution is removed at prescribed intervals and used for the analysis. For HPLC, the solution is extracted by using 6 ml DCM per 100 ml buffer solution with strong agitation for about 15 seconds, the solution in DCM part is separated and dried under nitrogen gas, and the extracted progesterone is dissolved in 1 ml acetonitrile and measured by HPLC. Alternatively, cumulative release of progesterone is measured directly from the aliquot of buffer via UV-Vis spectrophotometry.

The cumulative release of progesterone (and/or additional therapeutic agents) from the drug-eluting stent is assessed via a cumulative release plot that shows the release kinetics, with time plotted on a square-root scale.

### Example 8

### Evaluation of Restenosis for Coated Stent in Pig

Stent Preparation and Animal Selection. Five groups of three stents each are first prepared, the stents of each group having the same coating (or no coating), and each group having a distinct coating, varying in the drug composition. Each group has one of the following coatings: a polymer control stent, a bare stent, and three coated stents having a polymer layer and progesterone loaded at 0.1% wt, 1% wt, or 5% wt. Fifteen pigs are then selected and divided at random into groups containing three pigs each. The average pig weighs about 23 kg and prior to the experiment, the pigs are all kept in the same conditions and fed an experimental feed devoid of lipids. The pigs are also administered 300 mg/day of aspirin through their feed.

Each pig is systemically anesthetized with an injection of ketamine (22 mg/kg) and prepared for surgery. Next, an incision is made in the front of the neck at the midline exposing the carotid artery. A dose of heparin (300 U/kg) is injected into the artery of the pig at this time. A guide-wire is then inserted into the carotid artery through a small incision in the arterial wall. A guide catheter is then inserted and maneuvered to, and inside of, the left and right coronary artery. An appropriate site on the right coronary artery is selected with the use of a coronary artery angiography.

The appropriate stent is attached to a balloon catheter having a balloon capable of expanding to 10-20% larger than the diameter of the coronary artery. The balloon catheter is maneuvered to the site selected in the coronary artery and the balloon is inflated to its maximum size for 30 seconds at 4-12 atmospheric pressure to intentionally damage the coronary artery. After the balloon is deflated, the stent remains at the site. It is noted that, to block the coronary artery spasm following the blood vessel damage, nitroglycerin (200 ng) is continuously administered into the coronary artery through the guiding catheter. After the operation, a coronary artery angiography is conducted to observe the degree of damage to the coronary artery and the patency of the blood flow. The artery guide-wire is then removed and the slit in the carotid artery is ligated.

After 28 days, the pigs are again anesthetized and a guide-wire inserted as before. A dose of heparin (300 U/kg) is again injected via guide-wire into the artery. After confirming the patency of the blood vessels in the coronary artery, lethal amounts of pentothal and potassium chloride are injected via the guide catheter to induce euthanasia. The pig's heart is then removed through the thorax. The heart is then subjected to a perfusion-fixation procedure. Before sacrificing the animals, follow-up coronary angiography using OEC (GE medical, USA) is employed to determine the size of blood vessels and pictures taken before and after blood vessel damage are evaluated in order to determine the location and degree of arterial narrowing of the stented coronary segment.

The damaged portion of the artery along with an additional 2 cm region around the damaged site is removed from the heart. The specimen containing the stent is fixed using an embedding system (e.g., Technovit 7100, Kulzer, Germany). The specimen is then sliced into thin pieces with the use of a microtome equipped with a tungsten blade. Each slice is dyed with hematoxylin-eosin and elastic Van Gieson.

Each slice is then studied under a microscope. The slices are evaluated using the Schwartz scale. A quantitative and morphological analysis of the slices is conducted. In particular, the lumen area, internal elastic lamina area and external elastic area, intimal area, medial area, and the I/M ratio are determined. It is expected that the results will confirm that the coated stent loaded with progesterone will show a significantly reduced level of neointimal tissue volume at 28 days in a dose dependent manner when compared to the bare stent.

### Example 9

### Evaluation of the Effect of Various Compounds on Smooth Muscle Cell Proliferation

Progesterone and vitamin E are tested for their ability to prevent unitary (visceral) smooth muscle cell proliferation. Unitary smooth muscle cells are grown in appropriate culture media supplemented with dosages and compositions of progesterone; progesterone and vitamin E; progesterone and conjugated linoleic acid; or progesterone, vitamin E, and conjugated linoleic acid as described below for 0, 3, 7, 14, 21 and 28 days. For cell cultures grown longer than 3 days, culture medium is changed twice weekly, according to standard protocols in the art, and includes antibiotics and other standard additives as appropriate. The beginning cultures of unitary smooth muscle cells are provided at a sub-confluent density that will allow determination whether a given treatment causes either an increase or a decrease in the cell density.

Progesterone; progesterone and vitamin E; progesterone and conjugated linoleic acid; or progesterone, vitamin E, and conjugated linoleic acid are tested at different total doses in 11 different compositions. The doses are (expressed as the μg total of progesterone and vitamin E): 5, 10, 25, 50, 75, 100, 125, 150 and 200 μg. The compositions are: (1) progesterone, 0%; vitamin E, 100%; (2) progesterone, 25%; vitamin E, 75%; (3) progesterone, 50%; vitamin E, 50%; (4) progesterone, 75%; vitamin E, 25%; (5) progesterone, 100%; conjugated linoleic acid, 0%; (6) progesterone, 0%; conjugated linoleic acid, 100%; (7) progesterone, 25%; conjugated linoleic acid, 75%; (8) progesterone, 50%; conjugated linoleic acid, 50%; (9) progesterone, 75%; conjugated linoleic acid, 25%; (10) progesterone, 100%; conjugated linoleic acid, 0%. Progesterone, vitamin E, and conjugated linoleic acid; (11) progesterone, 50%, vitamin E, 25%, conjugated linoleic acid, 25%; are provided to cell cultures in ethanol, so appropriate amounts of ethanol are added to the culture medium as a control. In the cell cultures grown longer than 3 days, the indicated amounts of progesterone, vitamin E, or conjugated linoleic acid are supplied with each change of culture medium. Appropriate precautions are taken to prevent accelerated breakdown of these chemicals, including protection of the cell cultures from light. Optionally, supercritical fluid processing can be used instead of manufacturing and/or dissolving the progesterone in ethanol.

At each timepoint, triplicate samples are analyzed via cell viability, growth and density measurements. The rate of cell proliferation is assayed by counting the number of cells. Absolute cell density is measured with a Coulter Counter and can be counted from photographs. Cell viability is determined by trypan blue staining Rates of cell proliferation are also measured by determining the number of days to cell confluence in each treatment.

It is expected that one or more of the indicated dosages and compositions of progesterone, vitamin E, and conjugated linoleic acid will inhibit smooth muscle cell proliferation without causing cell mortality. This (these) dosage(s) and composition(s) will be considered for further testing in a larger cell-culture study.

### Example 10

### Evaluation of the Effect of Progesterone on Smooth Muscle Cell Proliferation

Progesterone was tested for its ability to prevent human aortic smooth muscle cell proliferation. Human aortic smooth muscle cells were grown in appropriate culture media supplemented with dosages and compositions of progesterone, as described below for 0, 3, 6, and 8 days. For cell cultures grown longer than 3 days, culture medium was changed twice weekly, according to standard protocols in the art, and included antibiotics and other standard additives as appropriate. The beginning cultures of human aortic smooth muscle cells were seeded into culture dishes at a low, sub-confluent density that allowed for linear growth rate of the untreated cells, thereby providing a basis for determining whether a given treatment caused either an increase or a decrease in smooth muscle cell growth rate.

Progesterone was initially dissolved in ethanol to attain a stock solution of 1 mg/ml. Ten μl (10 μg) of this progesterone stock 10 μg was added to each ml of cell culture medium prior to addition to the seeded cells. Addition of the progesterone-treated or control (untreated) medium to the cells was counted as day 0. In the cell cultures grown longer than 3 days, the indicated amounts of progesterone, was supplied with each third day change of culture medium. Cell cultures were maintained at 37° C. in an atmosphere containing 5% carbon dioxide. Appropriate precautions were taken to prevent accelerated breakdown, including protection of the cell cultures from light.

At each timepoint, triplicate samples were analyzed via growth measurements. The rate of cell proliferation was assayed by counting the number of cells across the 8 day growth period. Absolute cell density was measured with a Coulter Counter. Cell viability was determined by trypan blue staining.

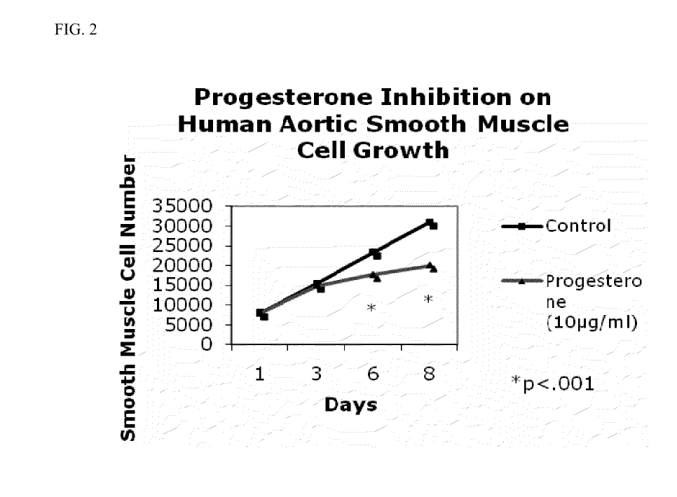

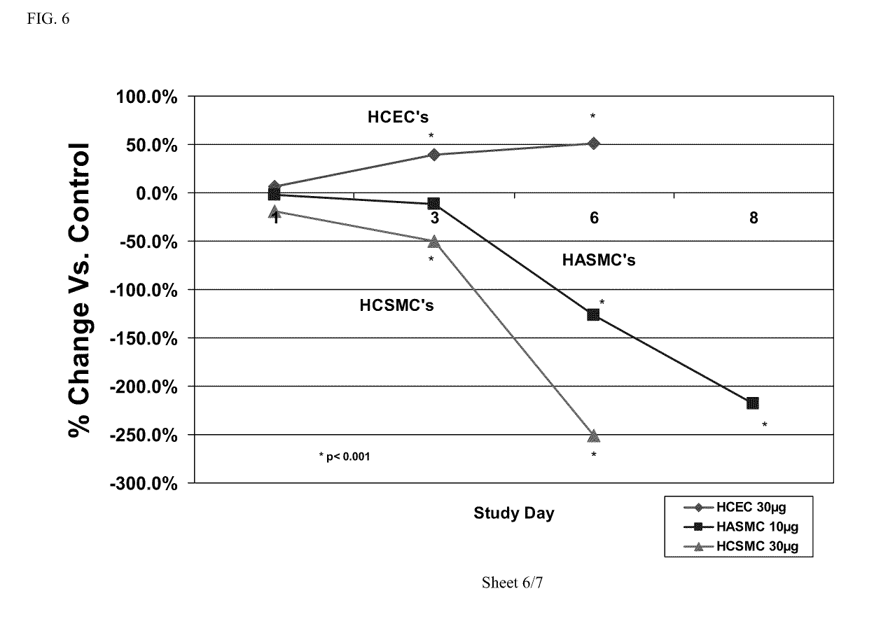

Results showed that the indicated dosage and composition of progesterone inhibited smooth muscle cell proliferation without causing cell mortality (see e.g., Table 1; FIG. 2; FIG. 6). At a p value of +<0.05, day 1 and 3 were not significantly different, while day 6 and 8 were significantly different. The above dosage and composition, along with additional dosages and formulations, will be considered for further testing in a larger cell-culture study, and also in a study that also tests for promotion of endothelial cell generation.

TABLE 1
Average number of cells per dish for control and progesterone
treated HASMC.

Day

| | 1 | 3 | 6 | 8 |
|---|---|---|---|---|
| Control | 8036 | 15467 | 23342 | 30916 |
| Progesterone | 7902 | 14871 | 17618 | 20018 |
| (10 μg/ml) | | | | |
| Probability* | 0.42 | 0.26 | 6.27E−06 | 1.15E−10 |

### Example 11

### Supercritical Fluid Used to Assist Loading of Polymer Microspheres for Sustained Release

An organic solvent-free method is used for encapsulating progesterone at high loadings within micron-sized inert latex polymer beads. This approach makes use of a polymeric surfactant to emulsify carbon dioxide into an aqueous latex suspension. Preformed 4 μm polystyrene (PS) microparticles surface-grafted with poly(N-vinylpyrrolidone) (PVP) are plasticized and swollen followed by rapid partitioning of progesterone into the polymer matrix. The as-prepared polystyrene beads is incorporated over 10% progesterone by weight. Dissolution experiments are also carried out to obtain the release profile of progesterone entrapped within the PVP/PS particles.

Styrene (99%), poly(N-vinylpyrrolidone) (Mw)) 40 kD), 2,2-azobisisobutyronitrile (AIBN), and progesterone is used. Absolute ethanol is used, and supercritical fluid grade CO₂ is passed through oxygen, water, and hydrocarbon traps prior to use. The poly(ethylene oxide)-block-poly(butylene oxide) (PEOb-PBO, tradename SAM 185) diblock surfactant is used. Nanopure water is used throughout. UV/vis spectra is taken.

The polymerization procedure is according to Yates et al. 2000 Langmuir 16, 4757-4760, except as otherwise noted. Briefly, poly(N-vinylpyrrolidone) (PVP, 1.5 g) is dissolved in 75 mL of ethanol. The ethanolic PVP solution is then heated to 70° C. in an oil bath while under a blanket of helium. In a separate flask, 0.25 g of AIBN is dissolved in 25 mL of styrene after removing the inhibitor by elution through an inhibitor removal column just prior. The AIBN in styrene solution is then added by syringe to the stirring PVP in ethanol. The styrene is polymerized for 24 h at 70° C. after which ethanol is removed. The final product is expected to be a highly monodisperse with a mean particle diameter of 3.6+/−0.2 μm.

PS particles (0.337 g) in the form of a dry powder are dispersed into 10 mL of Nanopure water. The resulting aqueous latex is loaded into a stainless steel variable-volume viewing cell along with 0.150 g of progesterone and 0.092 g of SAM 185. In a typical experiment, 3.5 g of CO₂ is added. The impregnation is carried out under continuous stirring at 25° C. and 310 bar for 24 h. It is important that CO2 pressure is released slowly and in a well controlled manner. The solvent is decanted, and the particles are resuspended in ethanol. Again, the sample is centrifuged followed by decanting to leave the progesterone-infused PVP grafted PS beads. The particles are air-dried overnight and then dried under vacuum for 4 h before study. Complete removal of ethanol is confirmed by ¹H NMR analysis for the progesterone-containing polymer beads in CD2Cl2. The ¹H NMR spectrum from the final material is expected to show three singlet peaks from progesterone (2.122, 1.220, and 0.680 ppm) and a broad PS matrix singlet corresponding to the phenyl protons of styrene (7.1 ppm). Based on relative integrated peak intensities, the matrix is expected to contain 10 wt % progesterone compared to styrene.

To study the release of progesterone from the loaded beads, 0.05 g of progesterone-loaded latex beads is suspended in 10 mL of ethanol. At various time increments, the sample is centrifuged and a UV/vis measurement is taken of the supernate. The amount of progesterone released after 8 h expected to be statistically equivalent (within 1.2%) to the level after 24 h (the absorbance at 24 h is thus taken as A∞).

Based on scanning electron microscopic evidence, controlled release materials formed using this approach starting with preexisting amorphous polymer microbeads are expected to exhibit no agglomeration. Further, it is expected that no particle growth, distortion, deformation, surface roughening, or foaming is observed compared with the unadulterated material. In addition, the CO₂ is expected to be readily removed by simply vaporizing under reduced pressure. Despite the low solubility of progesterone in CO₂, the latex beads are expected to readily load with 10% progesterone. The CO₂ in this process acts both as a swelling agent for the polymers in the latex bead and as a transfer agent for the progesterone. The poor solvation properties of CO₂, which limit potential applications for chemical reactions, offer an advantage for a transport medium. The progesterone is transported effectively by the supercritical phase of CO₂ with low mass transport barriers and readily deposits into the polymer matrix where it is more soluble.

As described above, CO₂-assisted impregnation is used to formulate polymer microspheres incorporating high levels of progesterone for controlled release. As is not generally the case with alternative routes in controlled release drug formulation, there is no exposure at any stage of the process to harmful organic solvents, mechanical stresses, or raised temperature. Any of the wide range of polymer latex suspensions can be used. As an example, biodegradable or bioerodible scaffolds based on poly(R-hydroxyacids) impregnated via this procedure have potential as drug eluting stents and drug delivery reservoirs.

### Example 12

### Progesterone Inhibits Human Infragenicular Arterial Smooth Muscle Cell Proliferation Induced by High Glucose and Insulin Concentrations

This example studies the effect of progesterone on vascular smooth muscle cells (VSMCs) exposed to various concentrations of glucose and insulin. Methods are according to 2002 J Vasc Surg 36, 833-838, except as otherwise noted.

Human infragenicular VSMCs are isolated from the tibial arteries of male patients with diabetes undergoing lower extremity amputation. Immunocytochemical studies with confocal microscopy are performed for progesterone receptor identification in these VSMCs. Cells are grown to subconfluence, followed by exposure to deprived media with various glucose (100 and 200 mg/dL) and insulin (no insulin and 100 ng/mL) concentrations. Cells are then additionally exposed to physiologic progesterone (10 ng/mL, progesterone group) and compared with a no-progesterone group. Cell count and methyl-3H-thymidine incorporation are used to determine cellular proliferation. Cell count with hemocytometry is performed on day 6. DNA synthesis as reflected through methyl-3H-thymidine incorporation is measured at 24 hours.

Immunocytochemical studies with confocal microscopy is expected to show cytosolic progesterone receptors. The noprogesterone group is expected to show a significant rise in cell count at all concentrations of glucose or insulin compared with the control group containing 100 mg/dL glucose concentration. The no-progesterone group is expected to show a significant rise in thymidine incorporation in the 100 mg/dL glucose-100 ng/mL insulin group and the 200 mg/dL glucose-100 ng/mL insulin group compared with the 100 mg/dL glucose group. In the cell count studies, progesterone is expected to significantly inhibited cellular proliferation in several settings. All cell groups cultured with insulin or an elevated glucose concentration are expected to show a significant antiproliferative effect when exposed to progesterone. With thymidine incorporation, progesterone is expected to show a similar antiproliferative effect in cells stimulated with glucose or insulin.

Expected significant reductions in cell proliferation as determined with both cell count and thymidine incorporation will show that progesterone is an inhibitor of VSMC proliferation induced by these in vitro models of hyperglycemia and hyperinsulinemia. Therefore, progesterone may have a protective role against the atherosclerotic changes.

### Example 13

### Comparison of Effects of Progesterone and Other Hormones on Glucocorticoid Inhibition in Astrocytes

This example explores study explored hormone inhibition in astrocytes. Glucocorticoids (GC), which are adrenal steroid hormones secreted during stress, can damage the hippocampus and impair its capacity to survive coincident neurological insults. The GC endangerment of the hippocampus is energetic in nature, as it can be prevented when neurons are supplemented with additional energy substrates. This energetic endangerment might arise from the ability of GC's to inhibit glucose transport into both hippocampal neurons and astrocytes. Thus arises the question as to whether the non-GC steroid progesterone inhibits glucose transport.

Methods are according to (1991) J Neurochem 57, 1422-1428, except as otherwise noted. Cells derived from fetal Sprague-Dawley rats on the 18^{th} day of gestation are cultured according to published methods (Horner et al. Neuroendocrinology 52, 57-64). Cells are then washed and counted. To obtain primary astrocyte cultures, approximately 7-8×10⁵ cells/well are plated in serum-supplemental medium into 96-well cluster dishes that are treated with 30 ug/ml of poly-D-lysine. Additional processing is according to Sapolsky et al. (1990) J. Neurosci. 10, 2897-2904. Cultures are either steroid free or exposed for 24 hours to dexamethasone (1 nM, 100 nM, or 10 μM); cortisol (1 μM), estrogen (1 μM), progesterone (1 μM), or testosterone (1 μM). The number of viable cells is determined by measuring intracellular activity of lactate dehydrogenase (LDH).

Results are expected to show features of classic steroid hormone action, i.e., that at least 4 hour of exposure are needed for inhibition. The inhibition is expected to be GC specific, with glucose transport expected to be inhibited by as little as 100 nM of synthetic GC, dexamethasone, or 1 uM of naturally occurring GC, cortisol, but is not expected to be inhibited by 1 μM of non-GC steroids such as estrogen, progesterone or testosterone.

Accordingly, progesterone is not expected to have the same inhibitory effect on glucose transport as GC steroids, such as dexamethasone. Based on such expectation, it is thought that progesterone, or a progesterone containing composition, may have a better therapeutic and/or safety effect if coated on or contained in a medical device implanted in the body of a subject.

### Example 14

### Evaluation of the Effect of Progesterone on Human Coronary Smooth Muscle Cell Proliferation and Human Coronary Endothelial Cell Growth

Progesterone was tested for its ability to prevent human coronary smooth muscle cell (HCSMC) proliferation and for its ability for promote human coronary endothelial cell (HSEC) growth.

Human coronary smooth muscle cells and human coronary endothelial cells were grown in appropriate culture media supplemented with dosages and compositions of progesterone, as described below for 1 and 3 days. In addition, cells will be grown for 5 and 7 days. For cell cultures grown longer than 3 days, culture medium is changed twice weekly, according to standard protocols in the art, and includes antibiotics and other standard additives as appropriate. The beginning cultures of human coronary smooth muscle cells were seeded into culture dishes at a low, sub-confluent density that allowed for linear growth rate of the untreated cells, thereby providing a basis for determining whether a given treatment caused either an increase or a decrease in smooth muscle cell growth rate. The beginning cultures of human coronary endothelial cells were seeded into culture dishes at a low, sub-confluent density that allowed for linear growth rate of the untreated cells, thereby providing a basis for determining whether a given treatment caused either an increase or a decrease in endothelial cell growth rate.

Progesterone was initially dissolved in ethanol to attain a progesterone stock solution of 1 mg/ml. Thirty (30) μg of progesterone stock was added to each ml of cell culture medium prior to addition to the seeded cells. Addition of the progesterone-treated or control (untreated) medium to the cells was counted as day 0. In the cell cultures grown longer than 3 days, the indicated amounts of progesterone, are supplied with each third day change of culture medium. Cell cultures were maintained at 37° C. in an atmosphere containing 5% carbon dioxide. Appropriate precautions were taken to prevent accelerated breakdown, including protection of the cell cultures from light.

Cultured human coronary endothelial and smooth muscle cells were obtained from commercial services. Progesterone was prepared as a stock solution using 100% ethanol. Dilutions were prepared using ethanol such that the total quantity of ethanolic drug volume added to any experimental culture was 10 μL. For proliferation and apoptosis studies, EC or SMC was seeded in multiple culture wells at a density of 5,000 cells/cm². Progesterone was added as 30 μg/ml, plus a control with zero amount of progesterone to triplicate sets of 4 EC or SMC cultures. Cell counts were conducted at days 1, 3, and 5 and will be conducted at day 7, using a Coulter counter. From these counts, the influence of the drug concentration on cell growth rate was calculated.

To evaluate the influence of progesterone on EC or SMC migration, cells are seeded on to a culture well surface and allowed to attain confluence. The cells are then wounded by removing the cells from half the surface. Following the wound, the cells are allowed to migrate into the wounded area. At the end of 7 days, the migration distance into that area is measured. These studies will be performed in the presence of either 0 or the 30 μg/ml drug concentration described above. The effect of these treatments will be compared statistically. Decreased cell proliferation and migration are hallmarks of SMC inhibition. On the other hand, increased EC proliferation and migration are hallmarks of endothelial healing.

To evaluate the influence of progesterone on apoptosis (cell death), EC or SMC are seeded at 5,000 cells/cm² and maintained in culture for 7 days. In contrast to the design for proliferation studies, apoptosis is analyzed on day 7 only. Apoptosis is performed using an immunostaining kit by Molecular Probes, Inc. This will determine whether progesterone might be acting to promote early death in either cell type. For application to DES, a decrease in EC apoptosis would be a favorable outcome while an increase in SMC apoptosis would be favorable.

Sampling and analysis of SMC is performed as follows. At each time point, triplicate samples are analyzed for growth measurements. The rate of cell proliferation is assayed by counting the number of cells across the 8 day growth period. Absolute cell density is measured with a Coulter Counter. Cell viability is determined by a live-dead cell apoptosis assay (Molecular Probes, Inc.).

Sampling and analysis of endothelial cells is performed as follows. At each time point, triplicate samples are analyzed for growth measurements. The rate of cell proliferation is assayed by counting the number of cells across the 8 day growth period. Absolute cell density is measured with a Coulter Counter. Cell viability is determined by a live-dead cell apoptosis assay (Molecular Probes, Inc.).

For apoptosis, additional endothelial cell and SMC culture plates are added to allow for assay of cell apoptosis and cell death. These cultures are run in parallel with the growth studies and receive the same treatment. The purpose is to determine whether any observed cell growth inhibition might be the result of cytotoxicity or a drug-induced increase in cell apoptosis.

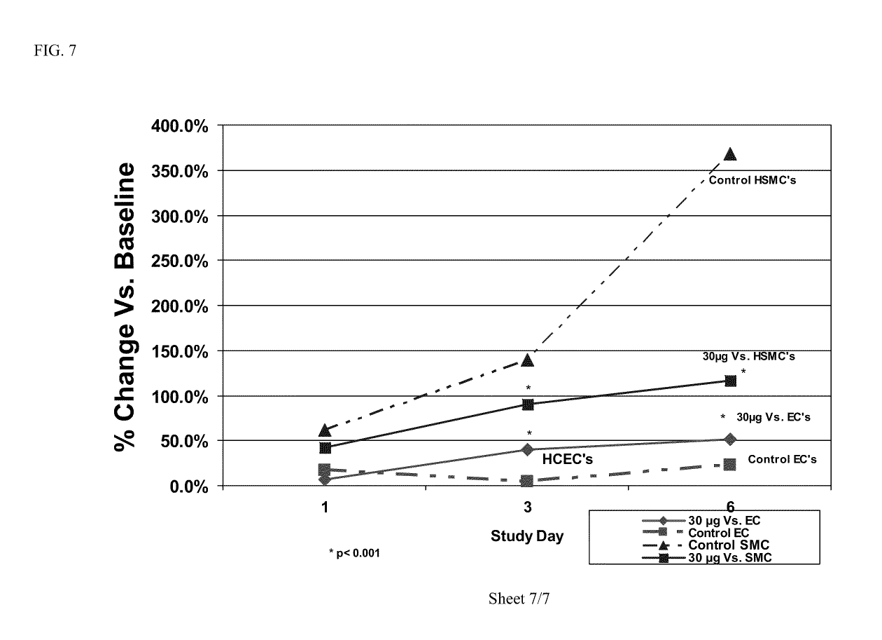

Results for HCSMC cell proliferation assays for are shown in Table 2, FIG. 6, and FIG. 7. Results for HCEC cell proliferation assays are shown in Table 3, FIG. 6, and FIG. 7.

TABLE 2
Average number of cells per dish for control and progesterone
treated HCSMC. Averages become significantly
different when the probability +<0.05.

| | Day | | | | |
|---|---|---|---|---|---|
| | 0 | 1 | 3 | 5 | 7 |
| | Average Number of Cells/dish | | | | |
| HCSMC Control | 5000 | 8071 | 11991 | 24413 | TBD |
| HCSMC Progesterone | 5000 | 7092 | 9492 | 10844 | TBD |
| (30 μg/ml) | | | | | |
| Probability (t-test) | N.S. | P < .001 | P < .001 | P < .001 | TBD |

TABLE 3
Average number of cells per dish for control and progesterone
treated HCEC. Averages become significantly different
when the probability +<0.05.

| | Day | | | | |
|---|---|---|---|---|---|
| | 0 | 1 | 3 | 5 | 7 |
| | Average Number of Cells/dish | | | | |
| HCEC Control | 5000 | 5876 | 5236 | 6124 | TBD |
| HCEC Progesterone | 5000 | 6207 | 7227 | 8658 | TBD |
| (30 μg/ml) | | | | | |
| Probability (t-test) | N.S. | N.S. | P < .001 | P < .001 | TBD |

The above study employs 30 μg of progesterone, which is three times that used in the previous study (see Example 10). This 30 μg dosage showed inhibition of SMC on the first day of treatment, which was unusually fast acting. In addition, while other drugs that inhibit SMC also typically inhibit EC's, 30 μg of progesterone did not. As shown above, progesterone actually promoted growth of the EC both on the first and third days.

The inhibition differences of SMC for days 1, 3, and 5 were statistically significant at P<0.001 (see e.g., Table 2). The growth difference for the day one was statistically significant at P<0.001 with a 12.1% difference in growth of progesterone treated SMC versus control. The growth difference for SMC day three was statistically significant at P<0.001 with a 20.8% difference in growth of progesterone treated SMC versus control. The growth difference for SMC day five was statistically significant at P<0.001 with a 53.7% difference in growth of progesterone treated SMC versus control. It is also noted that the rate of inhibition is shown to be increasing over time in the 30 μg progesterone treatment as well as the 10 μg progesterone treatment with a leveling off effect occurring around day 3 (see Example 10).

The growth differences for day one of the EC versus control was not statistically significant; however, there was a 5.3% increase in growth of the progesterone treated EC versus control (see e.g., Table 3). The growth difference for EC days 3 and 5 were statistically significant at P<0.001 with a 27.5% difference in growth of progesterone treated EC versus control at day 3; and a 29.3% difference in growth of progesterone treated EC versus control at day 5. It is also noted that the rate of growth is shown to be increasing over time in the 30 μg progesterone treatment, especially at and after day 3 (see e.g., EC, day 3, Table 3). While this was not measured with the 10 μg dosage, based on the findings for SMC, it is expected that rate of growth would increase over time at 10 μg progesterone as well.

Thus, for days 1, 3, and 5, the results indicate that plant progesterone (natural, plant based, gamma sterilized) at a concentration of 30 μg/ml inhibits SMC proliferation without causing cell mortality. At the time of filing, further SMC time point data is pending. As far as effect to EC, days 1, 3, and 5 showed that 30 μg/ml progesterone enhanced endothelial cell growth. At the time of filing, further EC time point data is pending. Furthermore, at the time of filing, apoptosis data is pending.

### INCORPORATION BY REFERENCE

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A drug eluting medical device comprising:
a medical device; and
a composition comprising progesterone;
wherein
the progesterone is present in a therapeutically effective amount; and
the progesterone is eluted in vivo.

2. The device of claim 1, comprising:
an eluting mechanism selected from the group consisting of a coating, reservoir, pore, duct, channel, chamber, side-port, and lumen;
wherein
the eluting mechanism is proximal to, distal to, lateral to, underneath, embedded within or on the device; and
the eluting mechanism elutes progesterone in vivo.

3. The device of claim 1, comprising:
at least one coating layer;
wherein
the at least one coating layer comprises the composition comprising progesterone; and
the at least one coating layer is formed on at least a portion of a surface of the medical device.

4. The device of claim 1, wherein the progesterone-containing composition further comprises at least one additional therapeutic agent selected from the group consisting of anti-platelets, anti-coagulants, anti-fibrins, anti-inflammatories, anti-thrombins, anti-proliferatives, anti-oxidants, anti-neoplastic, anti-cancer, anti-precancerous, and growth factors.

5. The device of claim 1, wherein the progesterone-containing composition comprises vitamin E; conjugated linoleic acid; or vitamin E and conjugated linoleic acid.

6. The device of claim 5, wherein the vitamin E comprises α-tocopherol or mycellized vitamin E.

7. The device of claim 3, wherein the coating layer further comprises a polymeric material.

8. The device of claim 2, wherein the progesterone-containing composition is introduced to the eluting mechanism in a process comprising compressed fluid, supercritical fluid processing, or supercritical carbon dioxide.

9. The device of claim 2, further comprising:
a barrier coating layer
wherein,
the barrier coating layer comprises a polymeric material and
the barrier coating layer controls elution of the progesterone-containing composition from the eluting mechanism.

10. The device of claim 1, wherein the drug eluting medical device is a drug eluting stent.

11. The device of claim 1, wherein the device is:
(i) configured to treat vulnerable plaque lesions;
(ii) configured to treat bifurcated lesions or ostial lesions;
(iii) configured for use in coronary, cardiac, saphenous vein grafts, peripheral carotid, neuro, gastrointestinal, gastroesophageal, gastroesophageal junction, prostate, uterine, cervix, vascular, organ, muscle, or body cavity applications; or
(iv) configured to treat cardiac allograft rejection or vasculopathy in a cardiac transplant recipient.

12. The device of claim 1, wherein the therapeutically effective amount is an anti-angiogenic, anti-thrombotic, anti-restenotic, vessel-relaxative, anti-oxidative, anti-cancer, anti-precancer, or anti-neoplastic effective amount, or a combination thereof.

13. The device of claim 1, wherein the progesterone comprises a natural progesterone, a USP grade progesterone, or a USP grade natural progesterone.

14. The device of claim 13, wherein the natural progesterone is a derivatized extract from a plant selected from Dioscorea or soybean.

15. The device of claim 14, wherein the natural progesterone is a derivatized extract from Dioscorea villosa, Dioscorea floribunda, Dioscorea macrostachya, or Dioscorea barbasco.

16. The device of claim 1, wherein in the progesterone-containing composition is comprised of a controlled-release delivery system.

17. The device of claim 16, wherein the controlled-release delivery system is selected from the group consisting of a microsphere, nanosphere, nanoscaffold, nanofiber, nanogel, hydrogel, liposome, polymersome, reservoir, and polymer micelle.

18. The device of claim 1, wherein progesterone and optional vitamin E, optional conjugated linoleic acid, or one or more optional additional therapeutic agents are eluted in series, in parallel, or in parallel and in series.

19. An anti-angiogenic, anti-thrombotic, anti-neoplastic, anti-cancer, anti-precancer, or anti-restenotic pharmaceutical formulation comprising:
(i) a therapeutically effective amount of progesterone;
(ii) vitamin E, conjugated linoleic acid, or vitamin E and conjugated linoleic acid; and
(iii) a pharmaceutically acceptable carrier;
wherein the therapeutically effective amount of progesterone is an anti-angiogenic, anti-thrombotic, anti-neoplastic, anti-cancer, anti-precancer, or anti-restenotic effective amount.

20. A method of treating a target tissue of a subject comprising:
introducing a drug eluting medical device of claim 1 to a target tissue of a subject in need thereof;
wherein progesterone is eluted from the introduced device in a therapeutically effective amount.
